(19) United States
(12) Patent Application Publication
(10) Pub. No.: US 2005/0089492 A1
(43) Pub. Date: Apr. 28, 2005

(54) Compositions containing at least one oil structured with at least one silicone-polyamide polymer, and at least one gelling agent and methods of using the same

(75) Inventors: Shaoxiang Lu, Plainsboro, NJ (US); Xavier Blin, Paris (FR); Jean Mondet, Aulnay-sous-Bois (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 10/935,352

(22) Filed: Sep. 8, 2004

Related U.S. Application Data
(63) Continuation of application No. 10/166,650, filed on Jun. 12, 2002, now abandoned.

(51) Int. Cl. A61K 7/06; A61K 7/11

(52) U.S. Cl. 424/70.12; 424/401

(57) ABSTRACT

The invention relates to a physiologically acceptable composition, in particular a cosmetic composition, containing at least one liquid fatty phase structured with at least one structuring polymer of the silicone-polyamide type, the polymer being solid at room temperature and soluble in said oil at a temperature of from 25 to 250° C., and at least one gelling agent for the liquid fatty phase, said liquid fatty phase containing at least one oil having an affinity with said structuring polymer and/or with said gelling agent, and the liquid fatty phase, the polymer and the gelling agent forming a physiologically acceptable medium. This composition may be in the form of a lipstick which is stable, which does not exude and whose application produces a glossy deposit with good staying power over time.

The present invention relates to a care and/or treatment and/or make-up composition for the skin, including the scalp, and/or for the lips of human beings, and/or for keratinous materials, such as keratinous fibres, containing a liquid fatty phase, structured with a specific polymer.

This composition can be stable over time and may be in the form of a stick of make-up such as lipstick, the application of which can produce a glossy deposit with good staying power or long-wearing properties.

It is common to find a structured, i.e., gelled and/or rigidified, liquid fatty phase in cosmetic or dermatological products; this is especially the case in solid compositions such as deodorants, lip balms, lipsticks, concealer products, eye shadows and cast foundations. This structuring may be obtained with the aid of waxes and/or fillers. Unfortunately, these waxes and fillers may have a tendency to make the composition matte, which may not always be desirable, in particular for a lipstick or an eye shadow. Consumers are always on the lookout for a lipstick in stick form which can deposit a film with good staying power or long wearing properties but which is also increasingly glossy.

The structuring of the liquid fatty phase may make it possible in particular to limit its exudation (or syneresis) from solid compositions, particularly in hot and humid areas and, furthermore, after deposition on the skin or the lips, to limit the migration of this phase into wrinkles and fine lines, a characteristic particularly desirable in a lipstick or eye shadow. The reason for this is that considerable migration of the liquid fatty phase, particularly when it is charged with colouring agents, may lead to an unpleasant appearance around the lips and the eyes, making wrinkles and fine lines particularly prominent. Consumers often state this migration as being a major drawback of conventional lipsticks and eye shadows. The term “migration” means movement of the composition beyond its initial site of application.

Gloss of a lipstick or other cosmetic is generally associated with the nature of the liquid fatty phase. Thus, it may be possible to reduce the amount of waxes and/or fillers in the composition in order to increase the gloss of a lipstick, but in that case the migration of the liquid fatty phase may increase. In other words, the amounts of waxes and of fillers required to prepare a stick of suitable hardness that does not exude at room temperature are a restricting factor on the gloss of the deposit.

To overcome at least one of these drawbacks, it has been envisaged replacing all or some of the waxes and/or fillers with polymers for structuring the liquid fatty phase, of the silicone-polyamide type. Unfortunately, the sticks obtained are not mechanically or thermally stable.

Furthermore, make-up compositions should have good staying power or long-wearing properties over time, i.e., little turning of or change in colour over time or a gradual or homogeneous change of the deposit over time. The turning of or change in colour of the deposit may be due, for lipsticks, to an interaction with saliva and, for foundations and eye shadows, to an interaction with the sweat and sebum secreted by the skin.

Furthermore, the majority of make-up or care compositions, when they are applied to the skin, eyelashes or lips, exhibit the disadvantage of transferring, that is to say of being at least partly deposited and leaving traces on certain substrates with which they may be brought into contact, in particular a glass, a cup, a cigarette, an item of clothing or the skin. This results in mediocre persistence of the applied film, requiring the regular renewal of the application of the composition, in particular a foundation or lipstick composition. In point of fact, it is the wish of users today to beautify their faces, including the lips, and their bodies while spending the least possible time doing so. Furthermore, the appearance of these unacceptable traces, in particular on blouse collars, can dissuade some women from using this type of make up.

The need thus remains for a composition which does not have at least one of the above drawbacks, which has good stability over time, even in hot atmosphere, and which produces a deposit on the skin or the lips that shows good staying power or long-wearing over time and has a glossy appearance. Furthermore, this composition can be easy to manufacture and can give the deposit a sensation of not drying out, both during application and over time.

One subject of the invention is a care and/or make-up and/or treatment composition for the skin and/or the lips of the face and/or for superficial body growths, i.e., keratinous materials, such as nails or keratinous fibres, which makes it possible to overcome at least one of the drawbacks mentioned above.

The inventors have found, surprisingly, that the use of at least one specific polymer combined with at least one gelling agent for a liquid fatty phase makes it possible to obtain a stick whose application to the lips produces a deposit, which can have noteworthy cosmetic properties. In particular, the deposit can be at least one of glossy, supple, comfortable and “migration-resistant”.

Furthermore, the composition can be stable over time at room temperature (25° C.) as well as high temperature (typically 47° C.). The term “stable” refers to a composition, in particular a stick that is hard and does not collapse over time at room temperature (25° C.) and at 47° C. for at least 1 month.

The invention applies not only to make-up products for the lips, such as lipsticks, lip glosses and lip pencils, but also to care and/or treatment products for the skin, including the scalp, and for the lips, such as antisun care products for the human face, the body or the lips, such as in stick form, make-up removing products for the skin of the face and body, make-up products for the skin, both of the human face and body, such as foundations optionally cast in stick or dish form, concealer products, blushers, eye shadows, face powders, transfer tattoos, body hygiene products (i.e., products which do not relate to the care, make-up, or treatment of keratin materials) such as deodorant, e.g., in stick form, shampoos, conditioners and make-up products for the eyes such as eyeliners, eye pencils and mascaras, e.g., in cake form, as well as make-up and care products for superficial body growths, for instance keratinous fibres such as the hair, the eyelashes, and the eyebrows or nails.

Another aspect of the invention is a composition comprising at least one liquid fatty phase comprising (i) at least one oil structured with at least one structuring polymer consisting of a polymer (homopolymer or copolymer) with a weight-average molecular mass ranging from 500 to 500 000, containing at least one moiety comprising:
at least one polyorganosiloxane group, consisting of from 1 to 1 000 organosiloxane units in the chain of the moiety or in the form of a graft, and
at least two groups capable of establishing hydrogen interactions, chosen from ester, amide, sulphonamide, carbamate, thiocarbamate, urea, thiourea, oxamido, guanamido and biguanidino groups, and combinations thereof, on condition that at least one of the groups is other than an ester group,
the polymer being solid at 25° C. and soluble in said oil at a temperature of from 25 to 250° C., and
(ii) at least one gelling agent for the liquid fatty phase,
said at least one liquid fatty phase comprising at least one oil having an affinity with said structuring polymer and/or with the gelling agent, and
the liquid fatty phase, the polymer and the gelling agent forming a physiologically acceptable medium.

The polymers used as structuring agents in the composition of the invention are polymers of the polyorganosiloxane type such as those described in documents U.S. Pat. No. 5,874,069, U.S. Pat. No. 5,919,441, U.S. Pat. No. 6,051,216 U.S. Pat. No. 5,981,680 and U.S. Pat. No. 6,051,216. Nevertheless, these documents specifically deal with deodorant and antiperspirant compositions.

The term “polymer” is understood to mean, within the meaning of the invention, a compound having at least 2 identical repeat units, preferably three identical units.

As used herein, the expression “gelling agent” means a compound that increases the liquid fatty phase viscosity and leads to a solid or flowable composition when introduced in said fatty phase. The gelling agent according to the present invention does not encompass waxes, in the sense that it is not waxy.

The gelling agent according to the present invention is preferably selected from the group consisting of agents that gel via chemical reticulation, in particular silicone resins; agents that gel via physical reticulation, in particular via molecular muddling, hydrogen interactions, sequences incompatibility or dipolar interactions, as well as liposoluble polymers having liquid crystal groups.

As used herein, the expression “at least one” means one or more and thus includes individual components as well as mixtures/combinations.

As used herein, the expression “affinity” means that the polymer and/or the gelling agent dissolves in said at least one oil, at a temperature higher than the melting temperature or the softening temperature of the structuring polymer and/or at a temperature higher than the melting temperature or the softening temperature of the gelling agent, in case the gelling agent is not in mineral form.

For the purposes of the invention, the expression “liquid fatty phase” means a fatty phase which is liquid at room temperature (25° C.) and atmospheric pressure (760 mmHg, i.e. 101 kPa), composed of one or more fatty substances that are liquid at room temperature, also referred to as oils, that are generally mutually compatible, i.e. forming a homogeneous phase macroscopically.

For the purposes of the patent application, the expression “structured liquid fatty phase” means that this structured phase does not run between the fingers and is at least thickened.

The polymer structuring the liquid fatty phase is solid at room temperature (25° C.) and atmospheric pressure (760 mm Hg) and soluble in said oil at a temperature from 25 to 250° C.

In the composition according to the present invention, the structuring polymer represents 0.5 to 80% by weight, preferably 2 to 60% by weight, more preferably 5 to 40% by weight, of the total weight of the composition.

Moreover, the structuring polymer preferably represents 0.1 to 50% by weight of the weight of the gelling agent together with the oil included in the liquid fatty phase.

The liquid fatty phase may contain at least one silicone oil.

The liquid fatty phase preferably contains at least 30%, and better still at least 40% by weight of silicone oil.

The composition of the invention can be in the form of a paste, a solid or a more or less viscous cream. It can be a single or multiple emulsion, such as an oil-in-water or water-in-oil emulsion or an oil-in-water-in-oil emulsion, or a water-in-oil-in-water emulsion, or a rigid or soft gel containing an oily continuous phase. For example, the liquid fatty phase can be the continuous phase of the composition. In one embodiment, the composition is anhydrous. In one embodiment, the composition is in a form cast as a stick or in a dish, for example solid, and further example, in the form of an oily rigid gel, such as an anhydrous gel, e.g., an anhydrous stick. In a further embodiment, the composition is in the form of an opaque or translucent rigid gel (depending on the presence or absence of pigments), and in a specific example, the liquid fatty phase forms the continuous phase. In one embodiment, the composition is chosen from molded and poured sticks.

The structuring of the liquid fatty phase can be modified depending on the nature of the structuring polymer and the gelling agent that are used, and may be such that a rigid structure in the form of a stick is obtained. When these sticks are colored, they make it possible, after application, to obtain a glossy deposit which does not migrate and/or which has good staying power, in particular of the colour over time, and/or does not transfer.

The composition of the invention can be a composition for the lips, such as a lipstick composition in stick form or a composition for the skin, such as a foundation.

Structuring Polymer

According to the invention, the polymers used as structuring agent may belong to the following two families:
1) polyorganosiloxanes comprising at least two groups capable of establishing hydrogen interactions, these two groups being located in the polymer chain; and/or
2) polyorganosiloxanes comprising at least two groups capable of establishing hydrogen interactions, these two groups being located on grafts or branches.

The polymer can dissolve in the oil or not. In case the oil does not establish hydrogen interactions with the structuring polymer, it may be necessary to heat the structuring polymer above its softening or melting temperature in order to break the intermolecular polymer hydrogen interactions. In case the oil does not establish hydrogen interactions with the structuring polymer, it may be otherwise necessary, before placing the structuring polymer in the presence of the oil according the invention, to dissolve said polymer beforehand in a solvent with hydrogen interaction capable of breaking the hydrogen interactions of the polymers, for instance C₂ to C₈ lower alcohols and especially ethanol, n-propanol or isopropanol. It is also possible to use these hydrogen interaction “breaking” solvents as co-solvents. These solvents may then be stored in the composition or may be removed by selective evaporation, which is well known to those skilled in the art.

In case the oil establishes hydrogen interactions with the structuring polymer, the structuring polymer can be dissolved in the oil at a temperature ranging from room temperature to its transition temperature (i.e. softening temperature or melting temperature).

The polymers comprising two groups capable of establishing hydrogen interactions in the polymer chain may be polymers comprising at least one moiety corresponding to the formula: 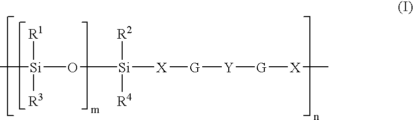
in which:
1) R¹, R², R³ and R⁴, which may be identical or different, represent a group chosen from:
   linear, branched or cyclic, saturated or unsaturated, C₁ to C₄₀ hydrocarbon-based groups, possibly containing in their chain one or more oxygen, sulphur and/or nitrogen atoms, and possibly being partially or totally substituted with fluorine atoms,
   C₆ to C₁₀ aryl groups, optionally substituted with one or more C₁ to C₄ alkyl groups,
   polyorganosiloxane chains possibly containing one or more oxygen, sulphur and/or nitrogen atoms;
2) the groups X, which may be identical or different, represent a linear or branched C₁ to C₃₀ alkylenediyl group, possibly containing in its chain one or more oxygen and/or nitrogen atoms;
3) Y is a saturated or unsaturated, C₁ to C₅₀ linear or branched divalent alkylene, arylene, cycloalkylene, alkylarylene or arylalkylene group, possibly comprising one or more oxygen, sulphur and/or nitrogen atoms, and/or bearing as substituent one of the following atoms or groups of atoms:
   fluorine, hydroxyl, C₃ to C₈ cycloalkyl, C₁ to C₄₀ alkyl, C₅ to C₁₀ aryl, phenyl optionally substituted with 1 to 3 C₁ to C₃ alkyl groups, C₁ to C₃ hydroxyalkyl and C₁ to C₆ aminoalkyl, or
4) Y represents a group corresponding to the formula: 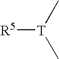
   in which
   T represents a linear or branched, saturated or unsaturated, C₃ to C₂₄ trivalent or tetravalent hydrocarbon-based group optionally substituted with a polyorganosiloxane chain, and possibly containing one or more atoms chosen from O, N and S, or T represents a trivalent atom chosen from N, P and Al, and
   R⁵ represents a linear or branched C₁ to C₅₀ alkyl group or a polyorganosiloxane chain, possibly comprising one or more ester, amide, urethane, thiocarbamate, urea, thiourea and/or sulphonamide groups, which may be linked to another chain of the polymer;
5) the groups G, which may be identical or different, represent divalent groups chosen from: 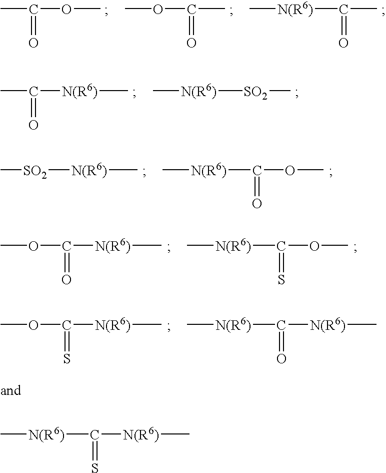
   in which R⁶ represents a hydrogen atom or a linear or branched C₁ to C₂₀ alkyl group, on condition that at least 50% of the groups R⁶ of the polymer represents a hydrogen atom and that at least two of the groups G of the polymer are a group other than: 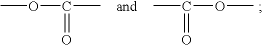
6) n is an integer ranging from 2 to 500 and preferably from 2 to 200, and m is an integer ranging from 1 to 1 000, preferably from 1 to 700 and better still from 6 to 200.

According to the invention, 80% of the groups R¹, R², R³ and R⁴ of the polymer are preferably chosen from methyl, ethyl, phenyl and 3,3,3-trifluoropropyl groups.

According to the invention, Y can represent various divalent groups, furthermore optionally comprising one or two free valencies to establish bonds with other moieties of the polymer or copolymer. Preferably, Y represents a group chosen from:
a) linear C₁ to C₂₀ and preferably C₁ to C₁₀ alkylene groups,
b) C₃₀ to C₅₆ branched alkylene groups possibly comprising rings and unconjugated unsaturations,
c) C₅-C₆ cycloalkylene groups,
d) phenylene groups optionally substituted with one or more C₁ to C₄₀ alkyl groups,
e) C₁ to C₂₀ alkylene groups comprising from 1 to 5 amide groups,
f) C₁ to C₂₀ alkylene groups comprising one or more substituents chosen from hydroxyl, C₃ to C₈ cycloalkane, C₁ to C₃ hydroxyalkyl and C₁ to C₆ alkylamine groups,
g) polyorganosiloxane chains of formula: 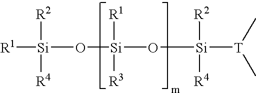
   in which R¹, R², R³, R⁴, T and m are as defined above, and
h) polyorganosiloxane chains of formula: 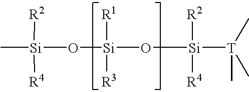
   in which R¹, R², R³, R⁴, T and m are as defined above.

The polyorganosiloxanes of the second family may be polymers comprising at least one moiety corresponding to formula (II): 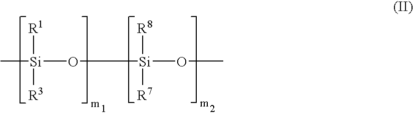
in which
R¹ and R³, which may be identical or different, are as defined above for formula (I),
R⁷ represents a group as defined above for R¹ and R³, or represents a group of formula —X—G—R⁹ in which X and G are as defined above for formula (I) and R⁹ represents a hydrogen atom or a linear, branched or cyclic, saturated or unsaturated, C₁ to C₅₀ hydrocarbon-based group optionally comprising in its chain one or more atoms chosen from O, S and N, optionally substituted with one or more fluorine atoms and/or one or more hydroxyl groups, or a phenyl group optionally substituted with one or more C₁ to C₄ alkyl groups,
R⁸ represents a group of formula —X—G—R⁹ in which X, G and R⁹ are as defined above,
m₁ is an integer ranging from 1 to 998, and
m₂ is an integer ranging from 2 to 500.

According to the invention, the polymer used as structuring agent may be a homopolymer, that is to say a polymer comprising several identical moieties, in particular moieties of formula (I) or of formula (II).

According to the invention, it is also possible to use a polymer consisting of a copolymer comprising several different moieties of formula (I), that is to say a polymer in which at least one of the groups R¹, R², R³, R⁴, X, G, Y, m and n is different in one of the moieties.

The copolymer may also be formed from several moieties of formula (II), in Which at least one of the groups R¹, R³, R⁷, R⁸, m₁ and m₂ is different in at least one of the moieties.

It is also possible to use a copolymer comprising at least one moiety of formula (I) and at least one moiety of formula (II), the moieties of formula (I) and the moieties of formula (II) possibly being identical to or different from each other.

According to one variant of the invention, it is also possible to use a copolymer furthermore comprising at least one hydrocarbon-based moiety comprising two groups capable of establishing hydrogen interactions, chosen from ester, amide, sulphonamide, carbamate, thiocarbamate, urea, thiourea oxamido, guanamido and biguanidino groups, and combinations thereof.

These copolymers may be block copolymers or grafted copolymers.

According to a first embodiment of the invention, the groups capable of establishing hydrogen interactions are amide groups of formulae —C(O)NH— and —HN—C(O)—.

In this case, the structuring agent may be a polymer comprising at least one moiety of formula (III) or (IV): 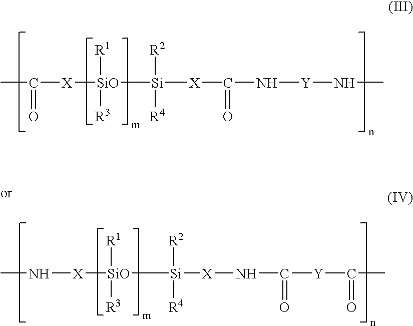
in which R¹, R², R³, R⁴, X, Y, m and n are as defined above.

Such a moiety may be obtained:
either by a condensation reaction between a silicone containing ,ω-carboxylic acid ends and one or more diamines, according to the following reaction scheme: 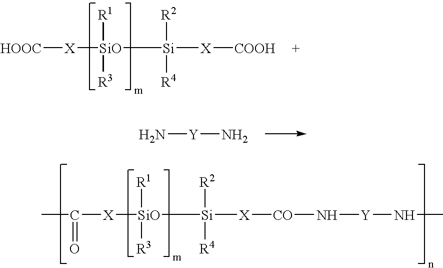
for by reaction of two molecules of -unsaturated carboxylic acid with a diamine according to the following reaction scheme:
   CH₂═CH—X¹—COOH+H₂N—Y—NH₂→
   followed by the addition of a siloxane to the ethylenic unsaturations, according to the following scheme: 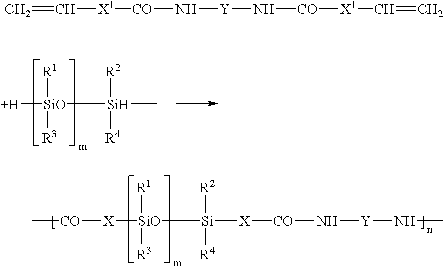
   in which X¹—(CH₂)₂— corresponds to X defined above and Y, R¹, R², R³, R⁴ and m are as defined above;
or by reaction of a silicone containing ,ω-NH₂ ends and a diacid of formula HOOC—Y—COOH according to the following reaction scheme:

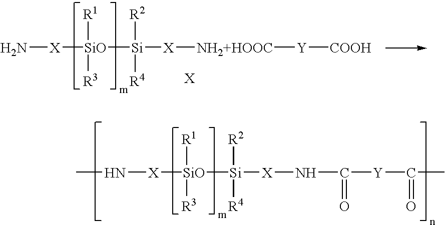

In these polyamides of formula (III) or (IV), m is preferably in the range from 1 to 700, more preferably from 15 to 500 and better still from 15 to 45, and n is in particular in the range from 1 to 500, preferably from 1 to 100 and better still from 4 to 25,
X is preferably a linear or branched alkylene chain containing from 1 to 30 carbon atoms and in particular 3 to 10 carbon atoms, and
Y is preferably an alkylene chain that is linear or branched or that possibly comprises rings and/or unsaturations, containing from 1 to 40 carbon atoms, in particular from 1 to 20 carbon atoms and better still from 2 to 6 carbon atoms, in particular 6 carbon atoms.

In formulae (III) and (IV), the alkylene group representing X or Y can optionally contain in its alkylene portion at least one of the following elements:
1°) 1 to 5 amide, urea or carbamate groups,
2°) a C₅ or C₆ cycloalkyl group, and
3°) a phenylene group optionally substituted with 1 to 3 identical or different C₁ to C₃ alkyl groups.

In formulae (III) and (IV), the alkylene groups may also be substituted with at least one element chosen from the group consisting of:
a hydroxyl group,
a C₃ to C₈ cycloalkyl group,
one to three C₁ to C₄₀ alkyl groups,
a phenyl group optionally substituted with one to three C₁ to C₃ alkyl groups,
a C₁ to C₃ hydroxyalkyl group, and
a C₁ to C₆ aminoalkyl group.

In these formulae (III) and (IV), Y may also represent: 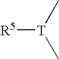
in which R⁵ represents a polyorganosiloxane chain and T represents a group of formula: 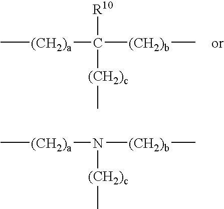
in which a, b and c are, independently, integers ranging from 1 to 10, and R¹⁰ is a hydrogen atom or a group such as those defined for R¹, R², R³ and R⁴.

In formulae (III) and (IV), R¹, R², R³ and R⁴ preferably represent, independently, a linear or branched C₁ to C₄₀ alkyl group, preferably a CH₃, C₂H₅, n-C₃H₇ or isopropyl group, a polyorganosiloxane chain or a phenyl group optionally substituted with one to three methyl or ethyl groups.

As has been seen previously, the polymer may comprise identical or different moieties of formula (III) or (IV).

Thus, the polymer may be a polyamide containing several moieties of formula (III) or (IV) of different lengths, i.e. a polyamide corresponding to the formula: 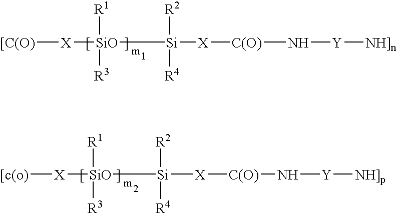
in which X, Y, n and R¹ to R⁴ have the meanings given above, m₁ and m₂, which are different, are chosen in the range from 1 to 1 000, and p is an integer ranging from 2 to 300.

In this formula, the moieties may be structured to form either a block copolymer, or a random copolymer or an alternating copolymer. In this copolymer, the moieties may be not only of different lengths, but also of different chemical structures, for example containing different groups Y. In this case, the copolymer may correspond to the formula: 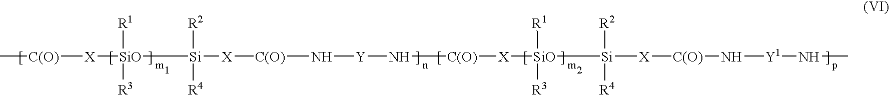
in which R¹ to R⁴, X, Y, m₁, m₂, n and p have the meanings given above and Y¹ is different from Y but chosen from the groups defined for Y. As previously, the various moieties may be structured to form either a block copolymer, or a random copolymer or an alternating copolymer.

In this first embodiment of the invention, the structuring polymer may also consist of a grafted copolymer. Thus, the polyamide containing silicone units may be grafted and optionally crosslinked with silicone chains containing amide groups. Such polymers may be synthesized with trifunctional amines.

In this case, the copolymer may comprise at least one moiety of formula: 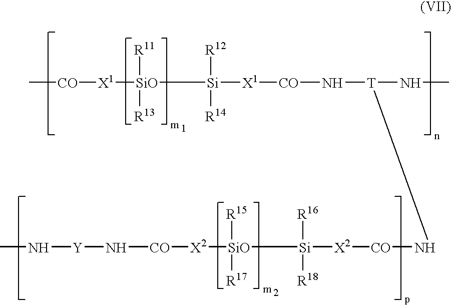
in which X¹ and X², which may be identical or different, have the meaning given for X in formula (I), n is as defined in formula (I), Y and T are as defined in formula (I), R¹¹ to R¹⁸ are groups chosen from the same group as R¹ to R⁴, m₁ and m₂ are numbers in the range from 1 to 1 000, and p is an integer ranging from 2 to 500.

In formula (VII), it is preferred that:
p is in the range from 1 to 25 and better still from 1 to 7,
R¹¹ to R¹⁸ are methyl groups,
T corresponds to one of the following formulae: 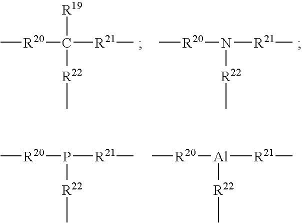
   in which R¹⁹ is a hydrogen atom or a group chosen from the groups defined for R¹ to R⁴, and R²⁰, R²¹ and R²² are, independently, linear or branched alkylene groups, and more preferably corresponds to the formula: 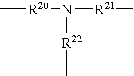
   in particular with R²⁰, R²¹ and R²² representing —CH₂—CH₂—,
m₁ and m₂ are in the range from 15 to 500 and better still from 15 to 45,
X¹ and X² represent —(CH₂)₁₀—, and
Y represents —CH₂—.

These polyamides containing a grafted silicone moiety of formula (VII) may be copolymerized with polyamide-silicones of formula (II) to form block copolymers, alternating copolymers or random copolymers. The weight percentage of grafted silicone moieties (VII) in the copolymer may range from 0.5% to 30% by weight.

According to the invention, as has been seen previously, the siloxane units may be in the main chain or backbone of the polymer, but they may also be present in grafted or pendent chains. In the main chain, the siloxane units may be in the form of segments as described above. In the pendent or grafted chains, the siloxane units may appear individually or in segments.

According to the invention, the preferred siloxane-based polyamides are:
polyamides of formula (III) in which m is from 15 to 50;
mixtures of two or more polyamides in which at least one polyamide has a value of m in the range from 15 to 50 and at least one polyamide has a value of m in the range from 30 to 50;
polymers of formula (V) with m₁ chosen in the range from 15 to 50 and m₂ chosen in the range from 30 to 500 with the portion corresponding to m₁ representing 1% to 99% by weight of the total weight of the polyamide and the corresponding portion m₂ representing 1% to 99% by weight of the total weight of the polyamide;
mixtures of polyamide of formula (III) combining
1) 80% to 99% by weight of a polyamide in which n is equal to 2 to 10 and in particular 3 to 6, and
2) 1% to 20% of a polyamide in which n is in the range from 5 to 500 and in particular from 6 to 100;
polyamides corresponding to formula (VI) in which at least one of the groups Y and Y¹ contains at least one hydroxyl substituent;
polyamides of formula (III) synthesized with at least one portion of an activated diacid (diacid chloride, dianhydride or diester) instead of the diacid;
polyamides of formula (III) in which X represents —(CH₂)₃— or —(CH₂)₁₀; and
polyamides of formula (III) in which the polyamides end with a monofunctional chain chosen from the group consisting of monofunctional amines, monofunctional acids, monofunctional alcohols, including fatty acids, fatty alcohols and fatty amines, such as, for example, octylamine, octanol, stearic acid and stearyl alcohol.

According to the invention, the end groups of the polymer chain may end with:
a C₁ to C₅₀ alkyl ester group by introducing a C₁ to C₅₀ monoalcohol during the synthesis,
a C₁ to C₅₀ alkylamide group by taking as stopping group a monoacid if the silicone is ,ω-diaminated, or a monoamine if the silicone is an ,ω-dicarboxylic acid.

According to one embodiment variant of the invention, it is possible to use a copolymer of silicone polyamide and of hydrocarbon-based polyamide, i.e. a copolymer comprising moieties of formula (III) or (IV) and hydrocarbon-based polyamide moieties. In this case, the polyamide-silicone moieties may be arranged at the ends of the hydrocarbon-based polyamide.

Polyamide-based structuring agents containing silicones may be produced by silylic amidation of polyamides based on fatty acid dimer. This approach involves the reaction of free acid sites existing on a polyamide as end sites, with organosiloxane-monoamines and/or organosiloxane-diamines (amidation reaction), or alternatively with oligosiloxane alcohols or oligosiloxane diols (esterification reaction). The esterification reaction requires the presence of acid catalysts, as is known in the art. It is desirable for the polyamide containing free acid sites, used for the amidation or esterification reaction, to have a relatively high number of acid end groups (for example polyamides with high acid numbers, for example from 15 to 20).

For the amidation of the free acid sites of the hydrocarbon-based polyamides, siloxane diamines with 1 to 300, more particularly 2 to 50 and better still 2, 6, 9.5, 12, 13.5, 23 or 31 siloxane groups, may be used for the reaction with hydrocarbon-based polyamides based on fatty acid dimers. Siloxane diamines containing 13.5 siloxane groups are preferred, and the best results are obtained with the siloxane diamine containing 13.5 siloxane groups and polyamides containing high numbers of carboxylic acid end groups. The reactions may be carried out in xylene to extract the water produced from the solution by azeotropic distillation, or at higher temperatures (about 180 to 200° C.) without solvent. Typically, the efficacy of the amidation and the reaction rates decrease when the siloxane diamine is longer, that is to say when the number of siloxane groups is higher. Free amine sites may be blocked after the initial amidation reaction of the diaminosiloxanes by reacting them either with a siloxane acid, or with an organic acid such as benzoic acid.

For the esterification of the free acid sites on the polyamides, this may be performed in boiling xylene with about 1% by weight, relative to the total weight of the reagents, of para-toluenesulphonic acid as catalyst.

These reactions carried out on the carboxylic acid end groups of the polyamide lead to the incorporation of silicone moieties only at the ends of the polymer chain.

It is also possible to prepare a copolymer of polyamide-silicone, using a polyamide containing free amine groups, by amidation reaction with a siloxane containing an acid group.

It is also possible to prepare a structuring agent based on a copolymer between a hydrocarbon-based polyamide and a silicone polyamide, by transamidation of a polyamide having, for example, an ethylene-diamine constituent, with an oligosiloxane- ,ω-diamine, at high temperature (for example 200 to 300° C.), to carry out a transamidation such that the ethylenediamine component of the original polyamide is replaced with the oligosiloxane diamine.

The copolymer of hydrocarbon-based polyamide and of polyamide-silicone may also be a grafted copolymer comprising a hydrocarbon-based polyamide backbone with pendent oligosiloxane groups.

This may be obtained, for example:
by hydrosilylation of unsaturated bonds in polyamides based on fatty acid dimers;
by silylation of the amide groups of a polyamide; or
by silylation of unsaturated polyamides by means of an oxidation, that is to say by oxidizing the unsaturated groups into alcohols or diols, to form hydroxyl groups that are reacted with siloxane carboxylic acids or siloxane alcohols. The olefinic sites of the unsaturated polyamides may also be epoxidized and the epoxy groups may then be reacted with siloxane amines or siloxane alcohols.

According to a second embodiment of the invention, the structuring polymer consists of a homopolymer or a copolymer comprising urethane or urea groups.

As previously, the polymer may comprise polyorganosiloxane moieties containing two or more urethane and/or urea groups, either in the backbone of the polymer or on side chains or as pendent groups.

The polymers comprising at least two urethane and/or urea groups in the backbone may be polymers comprising at least one moiety corresponding to the following formula: 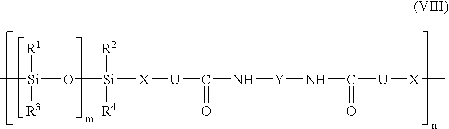
in which R¹, R², R³, R⁴, X, Y, m and n have the meanings given above for formula (I), and U represents —O— or —NH—, such that: 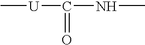
corresponds to a urethane or urea group.

In this formula (VIII), Y may be a linear or branched C₁ to C₄₀ alkylene group, optionally substituted with a C₁ to C₁₅ alkyl group or a C₅ to C₁₀ aryl group. Preferably, a —(CH₂)₆— group is used.

Y may also represent a C₅ to C₁₂ cycloaliphatic or aromatic group that may be substituted with a C₁ to C₁₅ alkyl group or a C₅ to C₁₀ aryl group, for example a radical chosen from the methylene-4,4-biscyclohexyl radical, the radical derived from isophorone diisocyanate, 2,4- and 2,6-tolylenes, 1,5-naphthylene, p-phenylene and 4,4′-biphenylenemethane. Generally, it is preferred for Y to represent a linear or branched C₁ to C₄₀ alkylene radical or a C₄ to C₁₂ cycloalkylene radical.

Y may also represent a polyurethane or polyurea block corresponding to the condensation of several diisocyanate molecules with one or more molecules of coupling agents of the diol or diamine type. In this case, Y comprises several urethane or urea groups in the alkylene chain.

It may correspond to the formula: 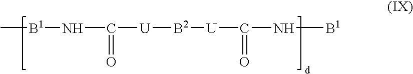
in which B¹ is a group chosen from the groups given above for Y, U is —O— or —NH— and B² is chosen from:
linear or branched C₁ to C₄₀ alkylene groups, which can optionally bear an ionizable group such as a carboxylic acid or sulphonic acid group, or a neutralizable or quaternizable tertiary amine group,
C₅ to C₁₂ cycloalkylene groups, optionally bearing alkyl substituents, for example one to three methyl or ethyl groups, or alkylene, for example the diol radical: cyclohexanedimethanol,
phenylene groups that may optionally bear C₁ to C₃ alkyl substituents, and
groups of formula: 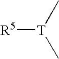
   in which T is a hydrocarbon-based trivalent radical possibly containing one or more hetero atoms such as oxygen, sulphur and nitrogen and R⁵ is a polyorganosiloxane chain or a linear or branched C₁ to C₅₀ alkyl chain.
T can represent, for example: 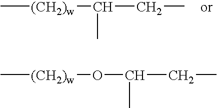
   with w being an integer ranging from 1 to 10 and R⁵ being a polyorganosiloxane chain. When Y is a linear or branched C₁ to C₄₀ alkylene group, the —(CH₂)₂— and —(CH₂)₆— groups are preferred.

In the formula given above for Y, d may be an integer ranging from 0 to 5, preferably from 0 to 3 and more preferably equal to 1 or 2.

Preferably, B² is a linear or branched C₁ to C₄₀ alkylene group, in particular —(CH₂)₂— or —(CH₂)₆— or a group: 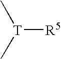
with R⁵ being a polyorganosiloxane chain.

As previously, the structuring polymer may be formed from silicone urethane and/or silicone urea moieties of different length and/or constitution, and may be in the form of block or random copolymers.

According to the invention, the silicone may also comprise urethane and/or urea groups no longer in the backbone but as side branches.

In this case, the polymer may comprise at least one moiety of formula: 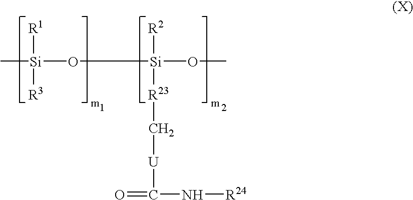
in which R¹, R², R³, m₁ and m₂ have the meanings given above for formula (I),
U represents O or NH,
R²³ represents a C₁ to C₄₀ alkylene group, optionally comprising one or more hetero atoms chosen from O and N, or a phenylene group, and
R²⁴ is chosen from linear, branched or cyclic, saturated or unsaturated C₁ to C₅₀ alkyl groups, and phenyl groups optionally substituted with one to three C₁ to C₃ alkyl groups.

The polymers comprising at least one moiety of formula (X) contain siloxane units and urea or urethane groups, and they may be used as gelling agents in the compositions of the invention.

The siloxane polymers may have a single urea or urethane group by branching or may have branches containing two urea or urethane groups, or alternatively they may contain a mixture of branches containing one urea or urethane group and branches containing two urea or urethane groups.

They may be obtained from branched polysiloxanes, comprising one or two amino groups by branching, by reacting these polysiloxanes with monoisocyanates.

As examples of starting polymers of this type containing amino and diamino branches, mention may be made of the polymers corresponding to the following formulae:

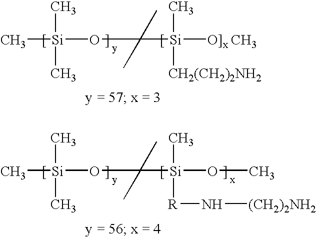

In these formulae, the symbol “/” indicates that the segments may be of different lengths and in a random order, and R represents a linear aliphatic group preferably containing 1 to 6 carbon atoms and better still 1 to 3 carbon atoms.

Such polymers containing branching may be formed by reacting a siloxane polymer, containing at least three amino groups per polymer molecule, with a compound containing only one monofunctional group (for example an acid, an isocyanate or an isothiocyanate) to react this monofunctional group with one of the amino groups and to form groups capable of establishing hydrogen interactions. The amino groups may be on side chains extending from the main chain of the siloxane polymer, such that the groups capable of establishing hydrogen interactions are formed on these side chains, or alternatively the amino groups may be at the ends of the main chain, such that the groups capable of hydrogen interaction will be end groups of the polymer.

As a procedure for forming a polymer containing siloxane units and groups capable of establishing hydrogen interactions, mention may be made of the reaction of a siloxane diamine and of a diisocyanate in a silicone solvent so as to provide a gel directly. The reaction may be performed in a silicone fluid, the resulting product being dissolved in the silicone fluid, at high temperature, the temperature of the system then being reduced to form the gel.

The polymers that are preferred for incorporation into the compositions according to the present invention are siloxane-urea copolymers that are linear and that contain urea groups as groups capable of establishing hydrogen interactions in the backbone of the polymer.

As an illustration of a polysiloxane ending with four urea groups, mention may be made of the polymer of formula: 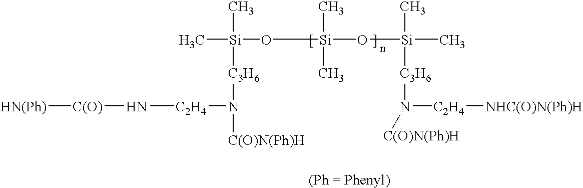
in which Ph is a phenyl group and n is a number from 0 to 300, in particular from 0 to 100, for example 50.

This polymer is obtained by reacting the following polysiloxane containing amino groups: 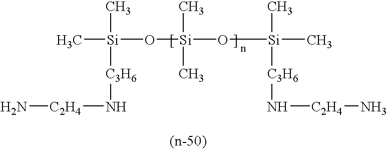
with phenyl isocyanate.

The polymers of formula (VIII) comprising urea or urethane groups in the chain of the silicone polymer may be obtained by reaction between a silicone containing ,ω-NH₂ or —OH end groups, of formula: 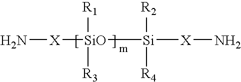
in which m, R¹, R², R³, R⁴ and X are as defined for formula (I) and a diisocyanate OCN—Y—NCO in which Y has the meaning given in formula (I); and optionally a diol or diamine coupling agent of formula H₂N—B²—NH₂ or HO—B²—OH, in which B² is as defined in formula (IX).

According to the stoechiometric proportions between the two reagents, diisocyanate and coupling agent, Y may have the formula (IX) with d equal to 0 or d equal to 1 to 5.

As in the case of the polyamide silicones of formula (II) or (III), it is possible to use in the invention polyurethane or polyurea silicones containing moieties of different length and structure, in particular moieties whose lengths differ by the number of silicone units. In this case, the copolymer may correspond, for example, to the formula: 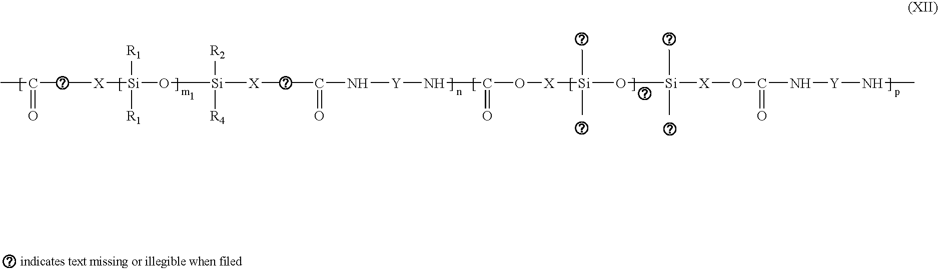
in which R¹, R², R³, R⁴, X, Y and U are as defined for formula (VIII) and m₁, m₂, n and p are as defined for formula (V).

Branched polyurethane or polyurea silicones may also be obtained using, instead of the diisocyanate OCN—Y—NCO, a triisocyanate of formula:

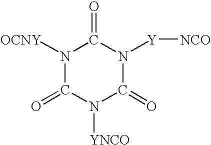

A polyurethane or polyurea silicone containing branches comprising an organosiloxane chain with groups capable of establishing hydrogen interactions is thus obtained. Such a polymer comprises, for example, a moiety corresponding to the formula: 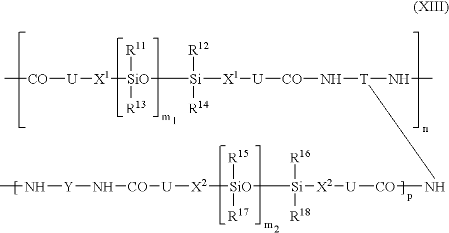
in which X¹ and X², which are identical or different, have the meaning given for X in formula (I) n is as defined in formula (I) Y and T are as defined in formula (I), R¹¹ to R¹⁸ are groups chosen from the same group as R¹ to R⁴, m₁ and m₂ are numbers in the range from 1 to 1 000, and p is an integer ranging from 2 to 500.

As in the case of the polyamides, this copolymer can also comprise polyurethane silicone moieties without branching.

In this second embodiment of the invention, the siloxane-based polyureas and polyurethanes that are preferred are:
polymers of formula (VIII) in which m is from 15 to 50;
mixtures of two or more polymers in which at least one polymer has a value of m in the range from 15 to 50 and at least one polymer has a value of m in the range from 30 to 50;
polymers of formula (XII) with m₁ chosen in the range from 15 to 50 and m₂ chosen in the range from 30 to 500 with the portion corresponding to m₁ representing 1% to 99% by weight of the total weight of the polymer and the portion corresponding to m₂ presenting 1% to 99% by weight of the total weight of the polymer;
mixtures of polymer of formula (VIII) combining
1) 80% to 99% by weight of a polymer in which n is equal to 2 to 10 and in particular 3to 6 and
2) 1% to 20% of a polymer in which n is in the range from 5 to 500 and in particular from 6 to 100
copolymers comprising two moieties of formula (VIII) in which at least one of the groups Y contains at least one hydroxyl substituent;
polymers of formula (VIII) synthesized with at least one portion of an activated diacid (diacid chloride dianhydride or diester) instead of the diacid;
polymers of formula (VIII) in which X represents —(CH₂)₃— or —(CH₂)₁₀—; and
polymers of formula (VIII) in which the polymers end with a multifunctional chain chosen from the group consisting of monofunctional amines, monofunctional acids, monofunctional alcohols, including fatty acids, fatty alcohols and fatty amines, such as, for example, octylamine, octanol, stearic acid and stearyl alcohol.

As in the case of the polyamides, copolymers of polyurethane or polyurea silicone and of hydrocarbon-based polyurethane or polyurea may be used in the invention by performing the reaction for synthesizing the polymer in the presence of an ,ω-difunctional block of non-silicone nature, for example a polyester, a polyether or a polyolefin.

As has been seen previously, the structuring polymer consisting of homopolymers or copolymers of the invention may contain siloxane moieties in the main chain of the polymer and groups capable of establishing hydrogen interactions, either in the main chain of the polymer or at the ends thereof, or on side chains or branches of the main chain. This may correspond to the following five arrangements: 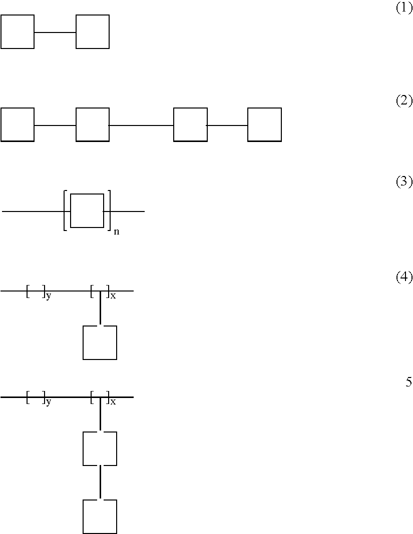
in which the continuous line is the main chain of the siloxane polymer and the squares represent the groups capable of establishing hydrogen interactions.

In case (1), the groups capable of establishing hydrogen interactions are arranged at the ends of the main chain.

In case (2), two groups capable of establishing hydrogen interactions are arranged at each of the ends of the main chain.

In case (3), the groups capable of establishing hydrogen interactions are arranged within the main chain in repeating moieties.

In cases (4) and (5), these are copolymers in which the groups capable of establishing hydrogen interactions are arranged on branches of the main chain of a first series of moieties that are copolymerized with moieties not comprising groups capable of establishing hydrogen interactions. The values n, x and y are such that the polymer has the desired properties in terms of an agent for gelling fatty phases based on silicone oil.

According to the invention, the structuring of the liquid fatty phase containing at least one silicone oil is obtained with the aid of one or more of the polymers mentioned above, in combination with solid particles with a hydrophobic surface.

In one embodiment, the structuring polymer is a silicone polyamide, also called a polyamide-modified silicone.

As examples of polymers that may be used, mention may be made of the silicone polyamides obtained in accordance with Examples 1 and 2 of document U.S. Pat. No. 5,981,680.

The at least one structuring polymer in the compositions of the invention may have a softening point greater than 50° C., such as from 65° C. to 190° C., and for example less than 150° C., and further such as from 70° C. to 130° C., and even further such as from 80° C. to 105° C. This softening point may be lower than that of structuring polymers used in the art which may facilitate the use of the at least one structuring polymer of the present invention and may limit the degradation of the liquid fatty phase. These polymers may be non-waxy polymers.

The softening point can be measured by a well-known method as “Differential Scanning Calorimetry” (i.e. DSC method) with a temperature rise of 5 to 10° C./min. They have good solubility in the silicone oils and produce macroscopically homogeneous compositions. Preferably, they have an average molecular mass from 500 to 200 000, for example from 1 000 to 100 000 and preferably from 2 000 to 30 000.

Further, an embodiment of the invention relates to a skin, lips, or keratinous fibres care, treatment, or make-up composition comprising at least one liquid fatty phase comprising (i) at least one oil structured with at least one above mentioned structuring polymer and (ii) at least one gelling agent for gelling the liquid fatty phase, said at least one liquid fatty phase comprising at least one oil having an affinity with said structuring polymer and/or with the gelling agent, and the liquid fatty phase, the polymer and the gelling agent forming a physiologically acceptable medium.

Additionally, an embodiment of the invention relates to a skin, lips, or keratinous fibers care or make-up composition comprising structured composition containing at least one liquid fatty phase comprising (i) at least one oil structured with at least one above mentioned structuring polymer (ii) at least one gelling agent for gelling the liquid fatty phase, and at least one coloring agent,
said at least one liquid fatty phase comprising at least one oil having an affinity with said structuring polymer and/or with the gelling agent, and
the liquid fatty phase, the polymer and the gelling agent forming a physiologically acceptable medium.

Another embodiment of the invention relates to a mascara, an eyeliner, a foundation, a lipstick, a blusher, a make-up-removing product, a make-up product for the body, an eyeshadow, a face powder, a concealer product, a shampoo, a conditioner, an antisun product or a care product for the lips, skin, or hair comprising a composition comprising at least one liquid fatty phase in the mascara, eyeliner, foundation, lipstick, blusher, make-up-removing product, make-up product for the body, eyeshadow, face powder, concealer product, shampoo, conditioner, antisun product or care product for the skin, lips, or hair which comprises at least one liquid fatty phase comprising (i) at least one oil structured with at least one above mentioned structuring polymer and (ii) at least one gelling agent for gelling the liquid fatty phase,
said at least one liquid fatty phase comprising at least one oil having an affinity with said structuring polymer and/or with the gelling agent, and
the liquid fatty phase, the polymer and the gelling agent forming a physiologically acceptable medium.

Another embodiment of the invention relates to a deodorant product or a care product for the skin or body comprising an anhydrous composition containing at least one liquid fatty phase comprising (i) at least one oil structured with at least one above mentioned structuring polymer and (ii) at least one polymeric gelling agent for gelling the liquid fatty phase,
said at least one liquid fatty phase comprising at least one oil having an affinity with said structuring polymer and/or with the gelling agent, and
the liquid fatty phase, the polymer and the gelling agent forming a physiologically acceptable medium.

Another embodiment of the invention relates to a lipstick composition comprising an anhydrous composition containing at least one liquid fatty phase comprising (i) at least one oil structured with at least one above mentioned structuring polymer and (ii) at least one gelling agent for gelling the liquid fatty phase,
said at least one liquid fatty phase comprising at least one oil having an affinity with said structuring polymer and/or with the gelling agent, and
the liquid fatty phase, the polymer and the gelling agent forming a physiologically acceptable medium.

Another embodiment of the invention relates to a method for care, make-up or treatment of keratin materials comprising applying to the keratin materials an anhydrous composition containing at least one liquid fatty phase comprising (i) at least one oil structured with at least one above mentioned structuring polymer and (ii) at least one gelling agent for gelling the liquid fatty phase,
said at least one liquid fatty phase comprising at least one oil having an affinity with said structuring polymer and/or with the gelling agent, and
the liquid fatty phase, the polymer and the gelling agent forming a physiologically acceptable medium.

Another embodiment of the invention relates to a method for care, make-up or treatment of keratinous fibres, lips, or skin comprising applying to the keratinous fibres, lips, or skin a composition comprising at least one liquid fatty phase comprising (i) at least one oil structured with at least one above mentioned structuring polymer and (ii) at least one gelling agent for gelling the liquid fatty phase,
said at least one liquid fatty phase comprising at least one oil having an affinity with said structuring polymer and/or with the gelling agent, and
the liquid fatty phase, the polymer and the gelling agent forming a physiologically acceptable medium.

Another embodiment of the invention relates to a method for providing an anhydrous composition having at least one property chosen from non-exudation, gloss, and comfortable deposit on keratin materials chosen from lips, skin, and keratinous fibres, comprising including in the composition at least one liquid fatty phase comprising (i) at least one oil structured with at least one above mentioned structuring polymer and (ii) at least one gelling agent for gelling the liquid fatty phase,
said at least one liquid fatty phase comprising at least one oil having an affinity with said structuring polymer and/or with the gelling agent, and
the liquid fatty phase, the polymer and the gelling agent forming a physiologically acceptable medium.

Another embodiment of the invention relates to a method of making up or caring for skin, lips or keratinous fibres comprising applying to the skin, lips, or keratinous fibres a structured composition containing at least one liquid fatty phase comprising (i) at least one oil structured with at least one above mentioned structuring polymer and (ii) at least one gelling agent for gelling the liquid fatty phase,
said at least one liquid fatty phase comprising at least one oil having an affinity with said structuring polymer and/or with the gelling agent, and the liquid fatty phase, the polymer and the gelling agent forming a physiologically acceptable medium.

Depending on the intended application, such as a stick, hardness of the composition may also be considered. The hardness of a composition may, for example, be expressed in gramforce (gf). The composition of the present invention may, for example, have a hardness ranging from 20 gf to 2000 gf, such as from 20 gf to 900 gf, and further such as from 20 gf to 600 gf.

This hardness is measured in one of two ways. A first test for hardness is according to a method of penetrating a probe into the composition and in particular using a texture analyzer (for example TA-XT2i from Rheo) equipped with an ebonite cylinder of height 25 mm and diameter 8 mm. The hardness measurement is carried out at 20° C. at the center of 5 samples of the composition. The cylinder is introduced into each sample of composition at a pre-speed of 2 mm/s and then at a speed of 0.5 mm/s and finally at a post-speed of 2 mm/s, the total displacement being 1 mm. The recorded hardness value is that of the maximum peak observed. The measurement error is ±50gf.

The second test for hardness is the “cheese wire” method, which involves cutting an 8.1 mm or preferably 12.7 mm in diameter stick composition and measuring its hardness at 20° C. using a DFGHS 2 tensile testing machine from Indelco-Chatillon Co. at a speed of 100 mm/minute. The hardness value from this method is expressed in grams as the shear force required to cut a stick under the above conditions. According to this method, the hardness of compositions according to the present invention which may be in stick form may, for example, range from 30 gf to 300 gf, such as from 30 gf to 250 gf, and further such as from 30 gf to 200 gf, for a 12.7 mm diameter stick.

The hardness of the composition of the present invention may be such that the compositions are self-supporting and can easily disintegrate to form a satisfactory deposit on a keratinous material. In addition, this hardness may impart good impact strength to the inventive compositions, which may be molded or cast, for example, in stick or dish form.

The skilled artisan may choose to evaluate a composition using at least one of the tests for hardness outlined above based on the application envisaged and the hardness desired. If one obtains an acceptable hardness value, in view of the intended application, from at least one of these hardness tests, the composition falls within the scope of the invention.

As is evident, the hardness of the composition according to the invention may, for example, be such that the composition is advantageously self-supporting and can disintegrate easily to form a satisfactory deposit on the skin and/or the lips and/or superficial body growths, such as keratinous fibres. In addition, with this hardness, the composition of the invention may have good impact strength.

According to the invention, the composition in stick form may have the behavior of a deformable, flexible elastic solid, giving noteworthy elastic softness on application. The compositions in stick form of the prior art do not have these properties of elasticity and flexibility.

Liquid Fatty Phase

For the purposes of the invention, the expression “liquid fatty phase” means a fatty phase which is liquid at room temperature (25° C.) and atmospheric pressure (760 mmHg, i.e. 101 kPa), composed of one or more fatty substances that are liquid at room temperature, also referred to as oils, that are generally mutually compatible, i.e. forming a homogeneous phase macroscopically. The expression “liquid fatty substance” means a non-aqueous liquid medium which is immiscible in all proportions with water, for example, a hydrocarbon-based compound comprising one or more carbon chains each containing at least 5 carbon atoms and possibly comprising at least one polar group chosen from carboxylic acid, hydroxyl, polyol, amine, amide, phosphoric acid, phosphate, ester, ether, urea, carbamate, thiol, thioether and thioester, a silicone compound optionally comprising carbon chains at the end or pendant, these chains optionally being substituted with a group chosen from fluoro, perfluoro, (poly)amino acid, ether, hydroxyl, amine, acid and ester groups; or a fluoro or perfluoro compound such as fluorohydrocarbons or perfluorohydrocarbons containing at least 5 carbon atoms, possibly comprising a hetero atom chosen from N, O, S and P and optionally at least one function chosen from ether, ester, amine, acid, carbamate, urea, thiol and hydroxyl groups.

The at least one liquid fatty phase comprising at least one oil has an affinity with the structuring polymer and/or with the gelling agent. The at least one oil, for example, may be chosen from polar oils and apolar oils including hydrocarbon-based liquid oils and oily liquids at room temperature. In one embodiment, the composition of the invention comprises at least one structuring polymer, at least one gelling agent and at least one apolar oil. The polar oils of the invention, for example, may be added to an apolar oil, the apolar oils acting in particular as co-solvent for the polar oils.

The liquid fatty phase of the composition may contain more than 30%, for example, more than 40%, of liquid oil(s) containing a group similar to that of the units of the structuring polymer, and for example from 50% to 100%. In one embodiment, the liquid fatty phase structured with a silicone-polyamide-type skeleton contains a high quantity, i.e., greater than 30%, for example greater than 40% relative to the total weight of the liquid fatty phase, or from 50% to 100%, of at least one apolar, such as hydrocarbon-based oil, silicone oils or mixtures thereof. For the purposes of the invention, the expression “hydrocarbon-based oil” means an oil essentially comprising carbon and hydrogen atoms, optionally with at least one group chosen from hydroxyl, ester, carboxyl and ether groups. With such a fatty phase, the at least one gelling agent may, for example, contain an amine, amide, urethane or silicone group.

For a liquid fatty phase structured with a polymer containing a partially silicone-based skeleton, this fatty phase may contain more than 30%, for example, more than 40%, relative to the total weight of the liquid fatty phase and, for example, from 50% to 100%, of at least one silicone-based liquid oil, relative to the total weight of the liquid fatty phase. In this embodiment, the at least one gelling agent may comprise a silicone group.

For example, the at least one polar oil useful in the invention may be chosen from:
hydrocarbon-based plant oils with a high content of triglycerides comprising fatty acid esters of glycerol in which the fatty acids may have varied chain lengths from C₄ to C₂₄, these chains possibly being chosen from linear and branched, and saturated and unsaturated chains; these oils can be chosen from, for example, wheat germ oil, corn oil, sunflower oil, karite butter, castor oil, sweet almond oil, macadamia oil, apricot oil, soybean oil, cotton oil, alfalfa oil, poppy oil, pumpkin oil, sesame oil, marrow oil, rapeseed oil, avocado oil, hazelnut oil, grape seed oil, blackcurrant seed oil, evening primrose oil, millet oil, barley oil, quinoa oil, olive oil, rye oil, safflower oil, candlenut oil, passion flower oil and musk rose oil; or alternatively caprylic/capric acid triglycerides such as those sold by Stearineries Dubois or those sold under the names Miglyol 810, 812 and 818 by Dynamit Nobel;
synthetic oils or esters of formula R₅COOR₆ in which R₅ is chosen from linear and branched fatty acid residues containing from 1 to 40 carbon atoms and R₆ is chosen from, for example, a hydrocarbon-based chain containing from 1 to 40 carbon atoms, on condition that R₅+R₆≧10, such as, for example, purcellin oil (cetostearyl octanoate), isononyl isononanoate, C₁₂-C₁₅ alkyl benzoates, isopropyl myristate, 2-ethylhexyl palmitate, isostearyl isostearate and alkyl or polyalkyl octanoates, decanoates or ricinoleates; hydroxylated esters such as isostearyl lactate and diisostearyl malate; and pentaerythritol esters;
synthetic ethers containing from 10 to 40 carbon atoms;
C₈ to C₂₆ fatty alcohols such as oleyl alcohol; and
C₈ to C₂₆ fatty acids such as oleic acid, linolenic acid or linoleic acid.

The at least one apolar oil according to the invention is chosen from, for example, silicone oils chosen from volatile and non-volatile, linear and cyclic polydimethylsiloxanes (PDMSs) that are liquid at room temperature; polydimethylsiloxanes comprising alkyl or alkoxy groups which are pendant and/or at the end of the silicone chain, the groups each containing from 2 to 24 carbon atoms; phenylsilicones such as phenyl trimethicones, phenyl dimethicones, phenyl trimethylsiloxy diphenylsiloxanes, diphenyl dimethicones, diphenyl methyldiphenyl trisiloxanes and 2-phenylethyl trimethylsiloxysilicates; hydrocarbons chosen from linear and branched, volatile and non-volatile hydrocarbons of synthetic and mineral origin, such as volatile liquid paraffins (such as isoparaffins and isododecane) or non-volatile liquid paraffins and derivatives thereof, liquid petrolatum, liquid lanolin, polydecenes, hydrogenated polyisobutene such as Parleam®, and squalane; and mixtures thereof. The structured oils, may be, in one embodiment, apolar oils, such as an oil or a mixture of hydrocarbon oils chosen from those of mineral and synthetic origin, chosen from hydrocarbons such as alkanes such as Parleam® oil, isoparaffins including isododecane, and squalane, and mixtures thereof.

In one embodiment, the liquid fatty phase comprises at least one non-volatile oil chosen from phenyisilicones, such as phenyl trimethicones.

In another embodiment, the liquid fatty phase contains at least one volatile oil. In another embodiment, the liquid fatty phase contains at least one volatile silicone oil.

In another embodiment, the viscosity of the oil according to the invention, in particular silicone oil, is less than 1000 cSt, and for example less than 100 cSt.

The term “volatile solvent” is understood to mean, within the meaning of the invention, any nonaqueous medium capable of evaporating on contact with the skin or lips in less than one hour at ambient temperature and atmospheric pressure. The volatile solvent or solvents of the invention are organic solvents and in particular volatile cosmetic oils which are liquid at ambient temperature and which have a nonzero vapor pressure, at ambient temperature and atmospheric pressure, ranging in particular from 10-3 to 300 mm of Hg (0.13 Pa to 40 000 Ps) and preferably greater than 0.03 mm of Hg (3.9 Pa).

The volatile solvents of the invention are preferably cosmetic oils chosen from oils which do not have a flashpoint, oils which have a flashpoint ranging from 40° C. to 100° C., and their mixtures, for the purpose of facilitating their processing. In addition, they advantageously exhibit a boiling point at atmospheric pressure of less than 220° C. and better still of less than 210° C., in particular ranging from 110 to 210° C.

According to the invention, these volatile solvents facilitate in particular the application of the composition to the skin, lips or superficial body growths. These solvents can be hydrocarbonaceous solvents, silicone solvents optionally comprising pendant alkyl or alkoxy groups or alkyl or alkoxy groups at the silicone chain end, or a mixture of these solvents. Preferably, these solvents are not monoalcohols comprising at least 7 carbon atoms.

Mention may be made, as volatile solvent which can be used in the invention, of linear or cyclic silicone oils having a viscosity at ambient temperature of less than 8 cSt and having in particular from 2 to 7 silicon atoms, these silicones optionally comprising alkyl or alkoxy groups having from 1 to 10 carbon atoms. Mention may in particular be made, as volatile silicone oils which can be used in the invention, of octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane, dodecamethylpentasiloxane and their mixtures.

Mention may be made, as other volatile solvent which can be used in the invention, of volatile hydrocarbonaceous oils having from 8 to 16 carbon atoms and their mixtures and in particular branched C₈-C₁₆ alkanes, such as C₈-C₁₆ and in particular C₈-C₁₃ isoalkanes (also known as isoparaffins), isodecane, isohexadecane and, for example, the oils sold under the trade names of Isopars L, E, H or G or of Permetyls, branched C₈-C₁₆ esters, such as isohexyl neopentanoate, and their mixtures. The volatile solvent is preferably chosen from volatile hydrocarbonaceous oils having from 8 to 16 carbon atoms and their mixtures, with the exception of Isopar M.

Volatile fluorinated solvents can also be used.

Use is preferably made of isododecane (Permetyls 99 A), C₈-C₁₆ isoparaffins (Isopars L, E and H), and their mixtures, optionally in combination with decamethyltetrasiloxane or with cyclopentasiloxane.

These volatile oils represent in particular a level by mass of 3 to 99.5% with respect to the total weight of the composition and in particular of 5 to 97.5%, preferably of 10 to 75% and better still of 15 to 45%. Generally, the amount of volatile solvent is used in an amount sufficient to produce transfer-free properties. This amount will be adjusted by a person skilled in the art according to the desired intensity of the transfer-free properties.

The liquid fatty phase, in one embodiment, contains at least one non-volatile oil chosen from, for example, hydrocarbon-based oils of mineral, plant and synthetic origin, synthetic esters or ethers, silicone oils and mixtures thereof.

In practice, the total liquid fatty phase can be, for example, present in an amount ranging from 1% to 99% by weight relative to the total weight of the composition, for example from 5% to 99%, 5% to 95.5%, from 10% to 80% or from 20% to 75%.

For the purposes of the invention, the expression “volatile solvent or oil” means any non-aqueous medium capable of evaporating on contact with the skin or the lips in less than one hour at room temperature and atmospheric pressure. The volatile solvent(s) of the invention is(are) organic solvents, such as volatile cosmetic oils that are liquid at room temperature, having a non-zero vapor pressure, at room temperature and atmospheric pressure, ranging in particular from 10-2 to 300 mmHg (1.33 to 40 000 Pa) and, for example, greater than 0.03 mmHg (4 Pa) and further example greater than 0.3 mmHg (40 Pa). The expression “non-volatile oil” means an oil which remains on the skin or the lips at room temperature and atmospheric pressure for at least several hours, such as those having a vapor pressure of less than 10-2 mmHg (1.33 Pa).

According to the invention, these volatile solvents may facilitate the staying power or long wearing properties of the composition on the skin, the lips or superficial body growths such as nails and keratinous fibres. The solvents can be chosen from hydrocarbon-based solvents, silicone solvents optionally comprising alkyl or alkoxy groups that are pendant or at the end of a silicone chain, and a mixture of these solvents.

The volatile oil(s), in one embodiment, can be present in an amount ranging from 0% to 95.5% relative to the total weight of the composition, such as from 2% to 75% or, for example, from 10% to 45%. This amount will be adapted by a person skilled in the art according to the desired staying power or long wearing properties.

The at least one liquid fatty phase of the composition of the invention may further comprises a dispersion of lipid vesicles. The composition of the invention may also, for example, be in the form of a fluid anhydrous gel, a rigid anhydrous gel, a fluid simple emulsion, a fluid multiple emulsion, a rigid simple emulsion or a rigid multiple emulsion. The simple emulsion or multiple emulsion may comprise a continuous phase chosen from an aqueous phase optionally containing dispersed lipid vesicles, or a fatty phase optionally containing dispersed lipid vesicles. In one embodiment, the composition has a continuous oily phase or fatty phase and is more specifically an anhydrous composition in, for example, a stick or dish form.

An anhydrous composition is one that has less than 10% water by weight, such as, for example, less than 5% by weight.

Gelling Agent

The composition of the invention -also contains at least one agent for gelling a liquid fatty phase. The gelling agent according to the present invention increases the liquid fatty phase viscosity and leads to a solid or flowable composition when introduced in said fatty phase. The gelling agent according to the present invention does not encompass waxes, in the sense that it is not waxy.

The at least one gelling agent may be chosen from gelling agents in polymeric form and gelling agents in mineral form.

In one embodiment, the at least one gelling agent is not soluble in an aqueous phase or in water.

The gelling agent according to the present invention is preferably selected from the group consisting of agents that gel via chemical reticulation and agents that gel via physical reticulation.

Gelling Agents that Gel via Chemical Reticulation

According to one embodiment, crosslinked elastomeric polyorganosiloxanes of three-dimensional structure, such as MQ silicone resins, polyalkylsesquioxanes, in particular polymethylsesquioxanes and resins reticulated via hydrosilylation, are preferred. These silicone resins can bear hydrophile groups, such as polyoxyethylene or copoly(oxyethylene/oxypropylene).

As polyorganosiloxanes which can be used in the invention, mention may be made of the crosslinked elastomeric polyorganosiloxanes described in application EP-A-0,295,886, the disclosure of which is incorporated herein by reference. According to that application, they are obtained by addition reaction and crosslinking, in the presence of a platinum-type catalyst, of at least:
(a) a polyorganosiloxane having at least two C₂ to C₆ lower alkenyl groups per molecule; and
(b) a polyorganosiloxane having at least two hydrogen atoms linked to a silicon atom per molecule. It is also possible to use the polyorganosiloxanes described in U.S. Pat. No. 5,266,321, the disclosure of which is incorporated by reference herein. According to that patent, they are chosen in particular from:
i) polyorganosiloxanes comprising R₂SiO and RSiO_{1.5} units and optionally R₃SiO_{0.5} and/or SiO₂ units in which the radicals R, independently of each other, are chosen from a hydrogen, an alkyl such as methyl, ethyl or propyl, an aryl such as phenyl or tolyl, an unsaturated aliphatic group such as vinyl, the weight ratio of the units R₂SiO to the units RSiO_{1.5} ranging from 1/1 to 30/1;
ii) polyorganosiloxanes which are insoluble and swellable in silicone-oil, obtained by addition of an polyorganohydrogenosiloxane (1) and of a polyorganosiloxane (2) having unsaturated aliphatic groups such that the amount of hydrogen or of unsaturated aliphatic groups in (1) and (2) respectively ranges from 1 to 20 mol % when the polyorganosiloxane is non-cyclic and from 1 to 50 mol % when the polyorganosiloxane is cyclic. Optionally, these polyorganosiloxanes can comprise from 1 to 40 oxyalkylene groups, such as oxypropylene and/or oxyethylene groups.

As examples of polyorganosiloxanes which can be used according to the invention, mention may be made of those sold or made under the names KSG6 from Shin-Etsu, Trefil E-505C or Trefil E-506C from Dow-Corning, Gransil from Grant Industries (SR-CYC, SR DMF10, SR-DC556) or those marketed in the form of preconstituted gels (KSG15, KSG17, KSG16, KSG18, KSG21 from Shin-Etsu, Gransil SR 5CYC gel, Gransil SR DMF 10 gel, Gransil SR DC556 gel, SF 1204 and JK 113 from General Electric. A mixture of these commercial products may also be used.

Gelling Agents that Gel via Physical Reticulation

Gelling agents that gel via physical reticulation, in particular via molecular muddling, hydrogen interactions, sequences incompatibility or dipolar interactions, as well as liposoluble polymers having liquid crystal groups, are preferred.

Gelling agents that gel via molecular muddling are polymers having high molecular weights, preferably higher than 500 000, such as silicone gums.

The silicone gum can correspond to the formula: 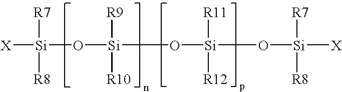
in which:
R₇, R₈, R₁₁ and R₁₂ are identical or different, and each is chosen from alkyl radicals comprising from 1 to 6 carbon atoms,
R₉ and R₁₀ are identical or different, and each is chosen from alkyl radicals comprising from 1 to 6 carbon atoms and aryl radicals,
X is chosen from alkyl radicals comprising from 1 to 6 carbon atoms, a hydroxyl radical and a vinyl radical,
n and p are chosen so as to give the silicone gum a viscosity of greater than 100 000 mPa.s, such as greater than 500 000 mPa.s.

In general, n and p can each take values ranging from 0 to 5 000, such as from 0 to 3 000.

Among the silicone gums which can be used according to the invention, mention may be made of those for which:
the substituents R₇ to R₁₂ and X represent a methyl group, p=0 and n=2 700, such as the product sold or made under the name SE30 by the company General Electric,
the substituents R₇ to R₁₂ and X represent a methyl group, p=0 and n=2 300, such as the product sold or made under the name AK 500 000 by the company Wacker,
the substituents R₇ to R₁₂ represent a methyl group, the substituent X represents a hydroxyl group, p=0 and n=2 700, as a 13% solution in cyclopentasiloxane, such as the product sold or made under the name Q2-1401 by the company Dow Corning,
the substituents R₇ to R₁₂ represent a methyl group, the substituent X represents a hydroxyl group, p=0 and n=2 700, as a 13% solution in polydimethylsiloxane, such as the product sold or made under the name Q2-1403 by the company Dow Corning, and
the substituents R₇, R₈, R₁₁, R₁₂ and X represent a methyl group and the substituents R₉ and R₁₀ represent an aryl group, such that the molecular weight of the gum is about 600 000, for instance the product sold or made under the name 761 by the company Rhone-Poulenc (Rhodia Chimie).

Gelling agents that gel the liquid fatty phase via hydrogen interactions are preferably chosen in the group consisting of:
amino silicones polymers having triazinyl groups or pyrimidinyl groups bound to amino groups of amino silicones as described in patent application EP 0 751 170, the disclosure of which is incorporated herein by reference,
non-silicone polyamides, ends of which bear ester or triamides functions, such as compounds described in patents and patent applications U.S. Pat. No. 5,783,657, U.S. Pat. No. 6,268,466, WO 01/95871, WO 00/40216, US 2002/0035237, and EP 1 068 856, the disclosure of which are incorporated herein by reference,
polyurethanes, such as compounds described in patent applications DE 10022247 and FR 2 814 365, the disclosure of which are incorporated herein by reference, and
vinyl and/or (meth)acrylic polymers bearing lateral groups that can create mutual hydrogen interactions, such as compounds described in patent application WO 93/01797, the disclosure of which is incorporated herein by reference.

Gelling agents that gel the liquid fatty phase via sequences incompatibility are preferably selected from the group consisting of:
block (di ou tri blocks) copolymers, such as polystyrene-silicone, or polyethylene-silicone, described in patents U.S. Pat. No. 6,225,390, U.S. Pat. No. 6,160,054, U.S. Pat. No. 6,174,968 and U.S. Pat. No. 6,225,390, the disclosures of which are incorporated herein by reference,
block or grafted copolymers comprising a silicone sequence and another sequence or graft that is polyvinyl or poly(meth)acrylic, such as those described in patents U.S. Pat. No. 5,468,477 et U.S. Pat. No. 5,725,882, the disclosures of which are incorporated herein by reference.
polymers or copolymers resulting from the polymerization or copolymerization of an ethylenic monomer, comprising one or more ethylenic, preferably conjugated, bonds (or dienes),
polymers or copolymers resulting from the polymerization or copolymerization of an ethylenic monomer, in particular use may be made of vinyl, acrylic or methacrylic copolymers which may be block copolymers, such as diblock or triblock copolymers, or even multiblock or starburst or radial copolymers. The at least one ethylenic gelling agent may comprise, for example, a styrene block (S), an alkylstyrene block (AS), an ethylene/butylene block (EB), an ethylene/propylene block (EP), a butadiene block (B), an isoprene block (I), an acrylate block (A), a methacrylate block (MA) or a combination of these blocks.
In one embodiment, a copolymer comprising at least one styrene block is used as gelling agent or ethylenic rheological agent. A triblock copolymer and in particular those of the polystyrene/polyisoprene or polystyrene/ polybutadiene type, such as those sold or made under the name “Luvitol HSB” by BASF and those of the polystyrene/copoly(ethylene-propylene) type or alternatively of the polystyrene/copoly(ethylene/butylene) type, such as those sold or made under the brand name “Kraton” by Shell Chemical Co. or Gelled Permethyl 99A by Penreco, may be used. Styrene-methacrylate copolymers can also be used.
As ethylenical gelling agent which can be used in the composition of the invention, mention may be made, for example, of Kraton (G1650 (SEBS), Kraton G1651 (SEBS), Kraton-G1652 (SEBS), Kraton G1657X (SEBS), Kraton G1701X (SEP), Kraton G1702X (SEP), Kraton G1726X (SEB), Kraton G1750X (EP) multiarm, Kraton G1765X (EP) multiarm, Kraton D-1101 (SBS), Kraton D-1102 (SBS), Kraton D-1107 (SIS), Gelled Permethyl 99A-750, Gelled Permethyl 99A-753-58 (mixture of starburst block polymer and triblock polymer), Gelled Permethyl 99A-753-59 (mixture of starburst block polymer and triblock polymer), Versagel 5970 and Versagel 5960 from Penreco (mixture of starburst polymer and triblock polymer in isododecane), and OS 129880, OS 129881 and OS 84383 from Lubrizol (styrene-methacrylate copolymer).
Di or triblocks such as polystyrene-copoly(ethylene/propylene) or polystyrene-copoly(ethylene/butylene) such as those described in patent applications WO 98/38981 and US 2002/0055562 are also included in the present invention.

Gelling agents that gel via dipolar interactions are preferably chosen from compounds describes in documents WO 01/30886 et U.S. Pat. No. 6,228,967, the disclosures of which are incorporated herein by reference. Ionized groups of said compounds, for example zwitterionic groups, create said dipolar interactions.

Gelling agents such as liposoluble polymers having liquid crystal groups are also preferred according to the present invention, especially liposoluble polymers whose backbone is silicone, vinyl and/or (meth)acrylic and that possess des lateral liquid crystal groups, in particular compounds described in patent application FR 2 816 503, the disclosure of which is incorporated herein by reference.

In another embodiment, the at least one gelling agent may be in mineral form with particle sizes that cause little or no light scattering. Thus, it may be possible to obtain a translucent or even transparent composition.

As modified clays which can be used, mention may be made of hectorites modified with an ammonium chloride of a C₁₀ to C₂₂ fatty acid, such as hectorite modified with distearyldimethylammonium chloride, also known as quatermium-18 bentonite, such as the products sold or made under the names Bentone 34 by the company Rheox, Claytone XL, Claytone 34 and Claytone 40 sold or made by the company Southern Clay, the modified clays known under the name quaternium-18 benzalkonium bentonites and sold or made under the names Claytone HT, Claytone GR and Claytone PS by the company Southern Clay, the clays modified with stearyldimethylbenzoylammonium chloride, known as steralkonium bentonites, such as the products sold or made under the names Claytone APA and Claytone AF by the company Southern Clay, and Baragel 24 sold or made by the company Rheox.

As other mineral gelling agents, which can be used in the invention, mention may be made of silica, such as fumed silica. The fumed silica may have a particle size, which may be nanometric to micrometric, for example ranging from about 5 nm to 200 nm.

The fumed silicas may be obtained by high-temperature hydrolysis of a volatile silicon compound in a hydrogen-oxygen flame, producing a finely divided silica. This process makes it possible to obtain hydrophilic silicas that have a large number of silanol groups at their surface. Such hydrophilic silicas are sold or made, for example, under the names “Aerosil 130®”, “Aerosil 200®”, “Aerosil 255®”, “Aerosil 300®” and “Aerosil 380®” by the company Degussa, and “CAB-O-SIL HS-55®”, “CAB-O-SIL EH-5®”, “CAB-O-SIL LM-130®”, “CAB-O-SIL MS-55®” and “CAB-O-SIL M-5®” by the company Cabot.

It is thus possible to chemically modify the surface of the hydrophilic silica by chemical reaction, producing a reduction in the number of silanol groups. The silanol groups can be replaced, for example, with hydrophobic groups: this then gives a hydrophobic silica. The hydrophobic groups may be:
trimethylsiloxyl groups, which are obtained in particular by treating fumed silica in the presence of hexamethyldisilazane. Silicas thus treated are known as “silica silylate” according to the CTFA (6th edition, 1995). They are sold or made, for example, under the references “Aerosil R812®” by the company Degussa and “CAB-O-SIL TS-530®” by the company Cabot;
dimethylsilyloxyl or polydimethylsiloxane groups, which are obtained in particular by treating fumed silica in the presence of polydimethylsiloxane or dimethyldichlorosilane. Silicas thus treated are known as “silica dimethyl silylate” according to the CTFA (6th edition, 1995). They are sold or made, for example, under the references “Aerosil R972®” and “Aerosil R974®” by the company Degussa, and “CAB-O-SIL TS-610®” and “CAB-O-SIL TS-720®” by the company Cabot;
groups derived from reacting fumed silica with silane alkoxides or siloxanes. These treated silicas are, for example, the products sold or made under the reference “Aerosil R805®” by the company Degussa.

According to the invention, hydrophobic silica, such as fumed silica, may be used as lipophilic gelling agent. The use of fumed silica makes it possible to obtain a translucent or even transparent composition, in particular in the form of a stick, which does not exude, in the absence of opacifying particles such as waxes, fillers and pigments (including nacres).

The at least one liposoluble gelling agent can allow the exudation of the composition to be limited and can allow its stability to be increased, while at the same time conserving the composition's glossy appearance, which is not possible with waxes such as those used conventionally in cosmetics and dermatology. These gelling agents can be used, for example, at concentrations of from 0.05% to 35% relative to the total weight of the composition, for example from 0.5% to 20% or from 1% to 10%.

Additional Additives

The composition of the invention can also comprise any additive usually used in the field under consideration, chosen in particular from dispersants such as poly(2-hydroxystearic acid), antioxidants, essential oils, preserving agents, fragrances, waxes, liposoluble polymers that are dispersible in the medium, fillers, neutralizing agents, cosmetic and dermatological active agents such as, for example, emollients, moisturizers, vitamins, essential fatty acids, sunscreens, and mixtures thereof. These additives may be present in the composition in a proportion of from 0% to 20% (such as from 0.01% to 20%) relative to the total weight of the composition and further such as from 0.01% to 10% (if present).

The composition of the invention can also contain, as an additive, an aqueous phase containing water that is optionally thickened or gelled with an aqueous-phase thickener or gelling agent and/or containing ingredients soluble in water. The water can represents from 0.01 to 50%, for example from 0.5 to 30% relative to the total weight of the composition.

In one embodiment, the composition comprises at least one surfactant or dispersing agent for dispersing water in the fatty phase, or for dispersing the fatty phase in an aqueous phase to obtain respectively an emulsion water-in-oil and an emulsion oil-in-water.

Needless to say, a person skilled in the art will take care to select the optional additional additives and/or the amount thereof such that the advantageous properties of the composition according to the invention are not, or are not substantially, adversely affected by the envisaged addition.

The composition according to the invention can be in the form of a tinted or non tinted dermatological composition or a care composition for keratin materials such as the skin, the lips and/or superficial body growths, in the form of an antisun composition or body hygiene composition in particular in the form of a deodorant product or make-up-removing product in stick form. It can be used in particular as a care base for the skin, superficial body growths or the lips (lip balms, for protecting the lips against cold and/or sunlight and/or the wind, or care cream for the skin, the nails or the hair). As defined herein, a deodorant product is personal hygiene product and does not relate to care, make-up or treatment of keratin materials, including keratinous fibres.

The composition of the invention may also be in the form of a colored make-up product for the skin, in particular a foundation, optionally having care or treating properties, a blusher, a face powder, an eye shadow, a concealer product, an eyeliner, a make-up product for the body; a make-up product for the lips such as a lipstick, optionally having care or treating properties; a make-up product for superficial body growths such as the nails or the eyelashes, in particular in the form of a mascara cake, or for the eyebrows and the hair, in particular in the form of a pencil.

Needless to say, the composition of the invention should be cosmetically or dermatologically acceptable, i.e. it should contain a non-toxic physiologically acceptable medium and should be able to be applied to the skin, superficial body growths or the lips of human beings. For the purposes of the invention, the expression “cosmetically acceptable” means a composition of pleasant appearance, odor, feel and taste.

The composition advantageously contains at least one cosmetic active agent and/or at least one dermatological active agent, i.e., an agent having a beneficial effect on the skin, lips or body growths and/or at least one coloring agent.

Coloring Agents

The composition may further comprise at least one coloring agent. The coloring agent according to the invention may be chosen from the lipophilic dyes, hydrophilic dyes, pigments and nacreous pigments (i.e., nacres) usually used in cosmetic or dermatological compositions, and mixtures thereof. This coloring agent is generally present in a proportion of from 0.01% to 50% relative to the total weight of the composition, such as from 0.5% to 40% and further such as from 5% to 30%, if it is present. In the case of a composition in the form of a free or compacted powder, the amount of coloring agent in the form of solid particles that are insoluble in the medium (nacres and/or pigments) may be up to 90% relative to the total weight of the composition.

The liposoluble dyes are, for example, Sudan Red, D&C Red 17, D&C Green 6, β-carotene, soybean oil, Sudan Brown, D&C Yellow 11, D&C Violet 2, D&C Orange 5, quinoline yellow or annatto. They can represent from 0.1% to 20% of the weight of the composition, for example, from 0.1% to 6% (if present). The water-soluble dyes are, for example, beetroot juice or methylene blue, and can represent up to 6% of the total weight of the composition.

The pigments may be white or coloured, goniochromatic or not, mineral and/or organic, and coated or uncoated. Among the mineral pigments which may be mentioned are titanium dioxide, optionally surface-treated, zirconium oxide, zinc oxide or cerium oxide, as well as iron oxide, chromium oxide, manganese violet, ultramarine blue, chromium hydrate and ferric blue. Among the organic pigments which may be mentioned are carbon black, pigments of D & C type, and lakes based on cochineal carmine or on barium, strontium, calcium or aluminium. The pigments can represent from 0.1% to 50%, such as from 0.5% to 40% and further such as from 2% to 30% relative to the total weight of the composition, if they are present.

The nacreous pigments may be chosen from white nacreous pigments such as mica coated with titanium or with bismuth oxychloride, coloured nacreous pigments such as titanium mica with iron oxides, titanium mica with, in particular, ferric blue or chromium oxide, titanium mica with an organic pigment of the type mentioned above, as well as nacreous pigments based on bismuth oxychloride. They can represent, for example, from 0.1% to 20% relative to the total weight of the composition, and further such as from 0.1% to 15%, if they are present.

In one embodiment, the coloring agent is a pigment (nacreous or not).

In another embodiment, the pigment is treated or not treated, and is preferably hydrophobic.

Waxes

The composition can optionally contain one or more waxes to improve the structuring in stick form, although this rigid form can be obtained in the absence of wax. For the purposes of the present invention, a wax is a lipophilic fatty compound that is solid at room temperature (25° C.) and atmospheric pressure (760 mmHg, i.e. 101 KPa), which undergoes a reversible solid/liquid change of state, having a melting point of greater than 40° C. and further such as greater than 55° C. and which may be up to 200° C., and having an anisotropic crystal organization in the solid state. The size of the crystals is such that the crystals diffract and/or scatter light, giving the composition a cloudy, more or less opaque appearance. By bringing the wax to its melting point, it is possible to make it miscible with oils and to form a microscopically homogeneous mixture, but on returning the temperature of the mixture to room temperature, recrystallization of the wax in the oils of the mixture is obtained. It is this recrystallization in the mixture which is responsible for the reduction in the gloss of the mixture. Thus, the composition advantageously contains little or no wax, and in particular less than 5% wax.

For the purposes of the invention, the waxes are those generally used in cosmetics and dermatology; they are, for example, of natural origin, for instance beeswax, carnauba wax, candelilla wax, ouricury wax, Japan wax, cork fibre wax, sugar cane wax, paraffin wax, lignite wax, microcrystalline waxes, lanolin wax, montan wax, ozokerites and hydrogenated oils such as hydrogenated jojoba oil as well as waxes of synthetic origin, for instance polyethylene waxes derived from the polymerization of ethylene, waxes obtained by Fischer-Tropsch synthesis, fatty acid esters and glycerides that are solid at 40° C., for example, at above 55° C., silicone waxes such as alkyl- and alkoxy-poly(di)methylsiloxanes and/or poly(di)methyl-siloxane esters that are solid at 40° C., for example, at above 55° C.

According to the invention, the melting point values correspond to the melting peak measured by the “Differential Scanning Calorimetry” method with a temperature rise of 5 or 10° C./min.

Liposoluble or Dispersible Polymers

The composition of the invention also can contain at least one polymer that is liposoluble or dispersible in the medium, other than the structuring polymer and the at least one rheological agent, and may have film-forming properties and may have, for example, an average molecular weight of from 500 to 1 000 000, such as from 1 000 to 500 000, and for example, further such as from 5 000 to 100 000, and even further such as from 5 000 to 20 000. This at least one liposoluble polymer may contribute towards increasing the viscosity and/or improving the staying power of the film. The at least one liposoluble polymer can have a softening point of not more than 30° C.

As examples of liposoluble polymers which can be used in the invention, mention may be made of: polyalkylenes, in particular polybutene, poly(meth)acrylates, alkylcelluloses with a linear or branched, saturated or unsaturated C₁ to C₈ alkyl radical, such as ethylcellulose and propylcellulose, silicone polymers that are compatible with the fatty phase, as well as vinylpyrrolidone (VP) copolymers, and mixtures thereof.

Vinylpyrrolidone copolymers, copolymers of a C₂ to C₃₀, such as C₃ to C₂₂ alkene, and combinations thereof, can be used. As examples of VP copolymers which can be used in the invention, mention may be made of VP/vinyl acetate, VP/ethyl methacrylate, butylated polyvinylpyrrolidone (PVP), VP/ethyl methacrylate/methacrylic acid, VP/eicosene, VP/hexadecene, VP/triacontene, VP/styrene or VP/acrylic acid/lauryl methacrylate copolymer.

Not only for the staying power properties but also for the feel and consistency properties of the film, the PVP/hexadecene copolymer having an average molecular weight of from 7 000 to 7,500 or alternatively the PVP/eicosene copolymer having an average molecular weight of from 8 000 to 9 000 can be used.

The liposoluble or dispersible polymers in the composition of the invention can be also used in an amount of from 0.01% to 20% (as active material) relative to the total weight of the composition, such as, for example, from 1% to 10%, if they are present.

The composition according to the invention may be manufactured by the known processes that are generally used in cosmetics or dermatology. It may be manufactured by the process which comprises heating the polymer at least to its softening point, adding the gelling agent(s), the coloring agent(s) and the additive(s) thereto and then mixing everything together until a clear, transparent solution is obtained. After reducing the temperature, the volatile solvent(s) is(are) then added to the mixture obtained. The homogeneous mixture obtained can then be cast in a suitable mould such as a lipstick mould or directly into the packaging articles (case or dish in particular).

Another aspect of the invention is a lipstick composition in stick form at least one liquid fatty phase comprising (i) at least one oil structured with at least one structuring polymer consisting of a polymer (homopolymer or copolymer) with a weight-average molecular mass ranging from 500 to 500 000, containing at least one moiety comprising:
at least one polyorganosiloxane group, consisting of from 1 to 1 000 organosiloxane units in the chain of the moiety or in the form of a graft, and
at least two groups capable of establishing hydrogen interactions, chosen from ester, amide, sulphonamide, carbamate, thiocarbamate, urea, thiourea, oxamido, guanamido and biguanidino groups, and combinations thereof, on condition that at least one of the groups is other than an ester group,
the polymer being solid at room temperature and soluble in said oil at a temperature of from 25 to 250° C., and
(ii) at least one gelling agent for the liquid fatty phase,
said at least one liquid fatty phase comprising at least one oil having an affinity with said
structuring polymer and/or with the gelling agent, and
the liquid fatty phase, the polymer and the gelling agent forming a physiologically acceptable medium.

The gelling agent and the structuring polymer can give the composition the appearance of a deformable elastic solid with a hardness ranging from 30 to 300 gf, such as 30 to 250 gf, and further such as 30 to 200 gf, even in the absence of wax. The hardness is measured by the “cheese-wire” method described above. The non-waxy polymer may be a polymer whose skeleton comprises units containing a hetero atom, as defined previously, and further may be a polyamide that may contain (an) alkyl end group(s) linked to the skeleton via an ester group.

An aspect of the invention is also a care, make-up or treatment cosmetic process for keratin materials of human beings, and in particular the skin, the lips and superficial body growths, comprising the application to the keratin materials of the composition, in particular the cosmetic composition, as defined above.

An aspect of the invention is also a combination (i) of at least one polymer consisting of a polymer (homopolymer or-copolymer) with a weight-average molecular mass ranging from 500 to 500 000, containing at least one moiety comprising:
at least one polyorganosiloxane group, consisting of from 1 to 1 000 organosiloxane units in the chain of the moiety or in the form of a graft, and
at least two groups capable of establishing hydrogen interactions, chosen from ester, amide, sulphonamide, carbamate, thiocarbamate, urea, thiourea, oxamido, guanamido and biguanidino groups, and combinations thereof, on condition that at least one of the groups is other than an ester group,
the polymer being solid at room temperature and soluble in said oil at a temperature of from 25 to 250° C., and
(ii) at least one gelling agent,
in a cosmetic composition or for the manufacture of a physiologically acceptable composition, to obtain a solid composition, such as a wax-free composition, which does not exude and/or which can produce a glossy and/or comfortable deposit on keratin materials,
said composition comprising a liquid fatty phase comprising at least one oil said liquid fatty phase having an affinity with said structuring polymer and/or with the gelling agent, and
the liquid fatty phase, the polymer and the gelling agent forming a physiologically acceptable medium.

The compositions of the present invention may also further comprise water, optionally thickened with an aqueous-phase thickener or gelled with a gelling agent and/or containing ingredients soluble in water.

The invention is illustrated in greater detail in the examples, which follow. The amounts are given as percentages by mass.

### EXAMPLE 1

### Lipstick

| | |
|---|---|
| silicone polyamide (polymerization degree 45) | 20% |
| phenyl trimethicone | 66.3% |
| (DC 556 ® DOW CORNING) | |
| hydrophobic silica (Aerosil R972) | 5% |
| pigments qsp | 100% |

Procedure

Silica gel: the gel was prepared, with stirring in a Rayneri stirrer at 60° C., using a hotplate, by introducing 5 g silica portionwise into 53 g DC 556.

Ground pigmentary material: the pigments were mixed with 13.3 g DC 556 heated to 60° C.; the mixture was ground three times in a three-roll mill.

The silicone polyamide was solubilized (or dissolved) at 100° C.-110° C. in the ground pigmentary material, followed by addition of the Silica gel. The whole mixture was mixed using a deflocculating turbomixer (Raynerie) and left stirring for 1H 30 min. The product obtained was then cast in molds for lipsticks in stick form.

The sticks of lipstick obtained had a diameter of 8.1 mm and a hardness of 135±2 gf measured using a “cheese wire”.

The stability of the compositions was tested using the test described herein. The composition was found to have good stability in that there was no exudation at room temperature (25° C.) and 47° C. for 1 month.

It is observed that a similar composition containing no silica only shows a hardness of 100±2 gf. This lipstick is easy to apply, non greasy and shiny. It does not exude.

### EXAMPLE 2

### Foundation

In phase A, ingredients are mixed well and ground with a Silverson homogenizer at a speed of 6000 rpm.

Separately the phase B1 ingredients are heated to 80 to 85° C. with stirring for 10-15 minutes or until dissolution of the siloxane-polyamide.

Phase A and B1 are then combined in the main beaker and mixed well at 70 to 75° C.

Phase B2 is added to the main beaker and is mixed until uniform.

Disteardimonium Hectorite is added to the main beaker and dispersed well before adding rest of phase B3 ingredients.

Phase C is heated to 70 to 75° C. in a separate side beaker. Emulsification is carried out by adding phase C to main beaker and homogenizing at medium/high speed.

The batch is cooled to room temperature with a paddle stirrer.

| PHASE | INCI Name | % W/W |
|---|---|---|
| A | Cyclopentasiloxane (and) dimethicone copolyol | 8.0 |
| | Polyglyceryl-4 isostearate (and) hexyl laurate | 3.5 |
| | (and) cetyl PEG/PPG-10/1 dimethicone | |
| | Treated pigments | 9.9 |
| B1 | Cyclopentasiloxane | 26.1 |
| | Silicone polyamide | 3.0 |
| B2 | Polytrap/cyclopentasiloxane | 1.0 |
| | MMA* Crosspolymer | 4.0 |
| | Nylon-12 | 1.0 |
| B3 | Preservative | 0.4 |
| | Disteardimonium Hectorite | 0.6 |
| | Propylene Carbonate | 0.2 |
| C | Water | 40.0 |
| | Magnesium Sulfate | 1.0 |
| | Preservatives | 0.7 |
| | Non ionic emulsifier | 0.5 |
| | TOTAL | 100.00 |

*MMA = methyl-methacrylate

The foundation provides a smooth application with excellent slip and cushion, and excellent transfer-resistance after drying. It further shows a very good water resistance.

### EXAMPLE 3

### Foundation

The composition is prepared as described in example 2.

| PHASE | INCI Name | % w/w |
|---|---|---|
| A | Cyclopentasiloxane (and) dimethicone copolyol | 8.0 |
| | Polyglyceryl-4 isostearate (and) hexyl laurate | 3.5 |
| | (and) cetyl PEG/PPG-10/1 dimethicone | |
| | Treated pigments | 9.9 |
| B1 | Volatile oil | 16.1 |
| | Silicone polyamide | 1.0 |
| | Silicone-Acrylates | 12.0 |
| B2 | Polytrap/cyclopentasiloxane | 1.0 |
| | MMA* Crosspolymer | 4.0 |
| | Nylon-12 | 1.0 |
| B3 | Preservative | 0.4 |
| | Disteardimonium Hectorite | 0.6 |
| | Propylene Carbonate | 0.2 |
| C | Water | 40.0 |
| | Magnesium Sulfate | 1.0 |
| | Preservatives | 0.2 |
| | Non ionic emulsifier | 0.5 |
| | TOTAL | 100.00 |

*MMA = methyl-methacrylate

The foundation provides a good application with cushion, good transfer-resistance after drying. The deposit shows a very good water resistant.

1-60. (canceled)

61. A make-up composition comprising at least one liquid fatty phase comprising
(i) at least one oil structured with at least one structuring polymer consisting of a polymer with a weight-average molecular mass ranging from 500 to 500,000, containing at least one moiety comprising:
at least one polyorganosiloxane group, consisting of from 1 to 1 000 organosiloxane units in the chain of the moiety or in the form of a graft, and
at least two groups capable of establishing hydrogen interactions, chosen from ester, amide, sulphonamide, carbamate, thiocarbamate, urea, thiourea, oxamido, guanamido and biguanidino groups, and combinations thereof, on condition that at least one of the groups is other than an ester group,
the polymer being solid at 25° C. and soluble in said oil at a temperature of from 25 to 250° C., and
(ii) at least one gelling agent for the liquid fatty phase,
wherein said at least one liquid fatty phase comprising at least one oil having an affinity with said structuring polymer and/or said gelling agent, and
wherein the liquid fatty phase, the structuring polymer and the gelling agent forming a physiologically acceptable medium.

62. A composition comprising at least one liquid fatty phase comprising
(i) at least one oil structured with at least one structuring polymer consisting of a polymer with a weight-average molecular mass ranging from 500 to 500,000, containing at least one moiety comprising:
at least one polyorganosiloxane group, consisting of from 1 to 1 000 organosiloxane units in the chain of the moiety or in the form of a graft, and
at least two groups capable of establishing hydrogen interactions, chosen from ester, amide, sulphonamide, carbamate, thiocarbamate, urea, thiourea, oxamido, guanamido and biguanidino groups, and combinations thereof, on condition that at least one of the groups is other than an ester group,
the polymer being solid at 25° C. and soluble in said oil at a temperature of from 25 to 250° C., and
(ii) at least one gelling agent for the liquid fatty phase selected from the group consisting of hectorites, fumed silica, and mixtures thereof,
wherein said at least one liquid fatty phase comprising at least one hydrocarbon oil having an affinity with said structuring polymer and/or said gelling agent, and
wherein the liquid fatty phase, the structuring polymer and the gelling agent forming a physiologically acceptable medium.

63. A composition comprising at least one liquid fatty phase comprising
(i) at least one hydrocarbon oil structured with at least one structuring polymer consisting of a polymer with a weight-average molecular mass ranging from 500 to 500,000, containing at least one moiety comprising:
at least one polyorganosiloxane group, consisting of from 1 to 1 000 organosiloxane units in the chain of the moiety or in the form of a graft, and
at least two groups capable of establishing hydrogen interactions, chosen from ester, amide, sulphonamide, carbamate, thiocarbamate, urea, thiourea, oxamido, guanamido and biguanidino groups, and combinations thereof, on condition that at least one of the groups is other than an ester group,
the polymer being solid at 25° C. and soluble in said oil at a temperature of from 25 to 250° C., and
(ii) at least one gelling agent for the liquid fatty phase,
wherein said at least one liquid fatty phase comprising at least one hydrocarbon oil having an affinity with said structuring polymer and/or said gelling agent, and
wherein the liquid fatty phase, the structuring polymer and the gelling agent forming a physiologically acceptable medium.

64. The composition according to claim 63, wherein the at least one gelling agent is a hectorite modified with an ammonium chloride of a C₁₀ to C₂₂ fatty acid.

65. The composition according to claim 64, wherein the at least one gelling agent is hectorite modified with distearyldimethylammonium chloride, quaternium-18 benzalkonium bentonites, stearyldimethylbenzoylammonium chloride, and mixtures thereof.

66. The composition according to claim 61, wherein the structuring polymer comprises at least one moiety corresponding to the formula: 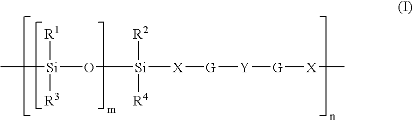
in which:
1) R¹, R², R³ and R⁴, which may be identical or different, represent one or more groups selected from the group consisting of:
linear, branched or cyclic, saturated or unsaturated, C₁ to C₄₀ hydrocarbon-based groups, possibly containing in their chain one or more oxygen, sulphur and/or nitrogen atoms, and optionally being partially or totally substituted with fluorine atoms,
C₆ to C₁₀ aryl groups, optionally substituted with one or more C₁ to C₄ alkyl groups, and
polyorganosiloxane chains possibly containing one or more oxygen, sulphur and/or nitrogen atoms;
2) the groups X, which may be identical or different, represent a linear or branched C₁ to C₃₀ alkylenediyl group, optionally containing in its chain one or more oxygen and/or nitrogen atoms;
3) Y is a saturated or unsaturated, C₁ to C₅₀ linear or branched divalent alkylene, arylene, cycloalkylene, alkylarylene or arylalkylene group, possibly comprising one or more oxygen, sulphur and/or nitrogen atoms, and/or bearing as substituent one of the following atoms or groups of atoms:
fluorine, hydroxyl, C₃ to C₈ cycloalkyl, C₁ to C₄₀ alkyl, C₅ to C₁₀ aryl, phenyl optionally substituted with 1 to 3 C₁ to C₃ alkyl groups, C₁ to C₃ hydroxyalkyl and C₁ to C₆ aminoalkyl, or
4) Y represents a group corresponding to the formula: 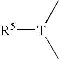
wherein
T represents a linear or branched, saturated or unsaturated, C₃ to C₂₄ trivalent or tetravalent hydrocarbon-based group optionally substituted with a polyorganosiloxane chain, and possibly containing one or more atoms selected from the group consisting of O, N and S, or T represents a trivalent atom chosen from N, P and Al, and
R⁵ represents a linear or branched C₁ to C₅₀ alkyl group or a polyorganosiloxane chain, optionally comprising one or more ester, amide, urethane, thiocarbamate, urea, thiourea and/or sulphonamide groups, which may optionally be linked to another chain of the polymer;
5) the groups G, which may be identical or different, represent one or more divalent groups selected from the group consisting of: 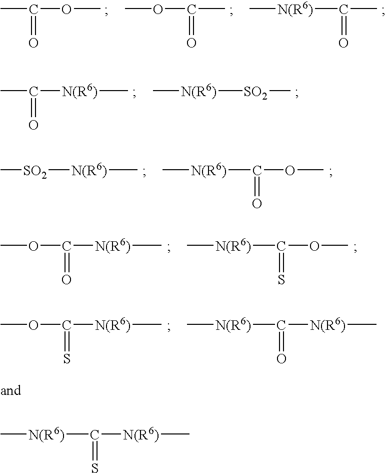
wherein R⁶ represents a hydrogen atom or a linear or branched C₁ to C₂₀ alkyl group, with the proviso that at least 50% of the groups R⁶ of the polymer represents a hydrogen atom and that at least two of the groups G of the polymer are a group other than: 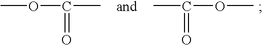
6) n is an integer ranging from 2 to 500, and m is an integer ranging from 1 to 1,000.

67. The composition according to claim 66, wherein n is an interger ranging from 2 to 200.

68. The composition according to claim 66, wherein m is an integer ranging from 1 to 700.

69. The composition according to claim 66, wherein m is an integer ranging from 6 to 200.

70. The composition according to claim 61, wherein the liquid fatty phase comprises a hydrocarbon oil structured with the at least one structuring polymer.

71. Composition according to claim 66, wherein Y represents a group selected from the group consisting of:
a) linear C₁ to C₂₀ alkylene groups,
b) C₃₀ to C₅₆ branched alkylene groups possibly comprising rings and unconjugated unsaturations,
c) C₅-C₆ cycloalkylene groups,
d) phenylene groups optionally substituted with one or more C₁ to C₄₀ alkyl groups,
e) C₁ to C₂₀ alkylene groups comprising from 1 to 5 amide groups,
f) C₁ to C₂₀ alkylene groups comprising one or more substituents selected from the group consisting of hydroxyl, C₃ to C₈ cycloalkane, C₁ to C₃ hydroxyalkyl and C₁ to C₆ alkylamine groups,
g) polyorganosiloxane chains of formula: 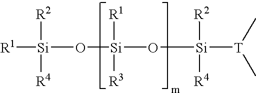
in which R¹, R², R³, R⁴, T and m are as defined above, and
h) polyorganosiloxane chains of formula: 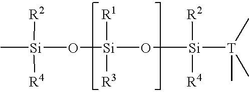
in which R¹, R², R³, R⁴, T and m are as defined above.

72. The composition according to claim 71, wherein Y represents C₁ to C₁₀ alkylene group.

73. The composition according to claim 61, wherein the structuring polymer comprises at least one moiety corresponding to formula (II): 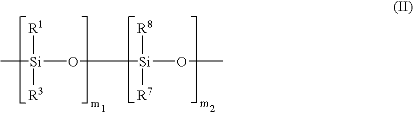
wherein
R¹ and R³, which may be identical or different, are, independently, linear or branched alkylene groups,
R⁷ represents a group as defined above for R¹ and R³, or represents a group of formula —X—G—R⁹;
wherein X represents a linear or branched C₁ to C₃₀ alkylenediyl group, possibly containing in its chain one or more oxygen and/or nitrogen atoms; and
G represents divalent groups selected from the group consisting of: 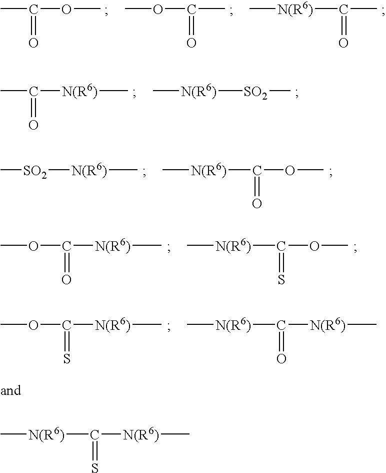
in which R⁶ represents a hydrogen atom or a linear or branched C₁ to C₂₀ alkyl group, on condition that at least 50% of the groups R⁶ of the polymer represents a hydrogen atom and that at least two of the groups G of the polymer are a group other than: 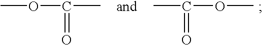
and
R⁹ represents a hydrogen atom or a linear, branched or cyclic, saturated or unsaturated, C₁ to C₅₀ hydrocarbon-based group optionally comprising in its chain one or more atoms selected from the group consisting of O, S and N, optionally substituted with one or more fluorine atoms and/or one or more hydroxyl groups, or a phenyl group optionally substituted with one or more C₁ to C₄ alkyl groups,
R⁸ represents a group of formula —X—G—R⁹ in which X, G and R⁹ are as defined above,
m₁ is an integer ranging from 1 to 998, and
m₂ is an integer ranging from 2 to 500.

74. The composition according to claim 71, wherein the polymer comprises at least one moiety of formula (III) or (IV): 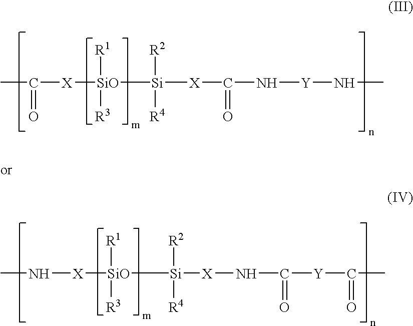
wherein
R¹, R², R³, R⁴, are, independently, linear or branched alkylene groups X represents —(CH₂)₁₀—,
Y represents —CH₂—,
m is an integer ranging from 1 to 1,000; and
n is an integer ranging from 2 to 500.

75. The composition according to claim 66, wherein X and/or Y represent an alkylene group containing in its alkylene portion at least one of the following elements:
1°) 1 to 5 amide, urea or carbamate groups,
2°) a C₅ or C₆ cycloalkyl group, and
3°) a phenylene group optionally substituted with 1 to 3 identical or different C₁ to C₃ alkyl groups, and/or substituted with at least one element selected from the group consisting of a hydroxyl group, a C₃ to C₈ cycloalkyl group, one to three C₁ to C₄₀ alkyl groups, a phenyl group optionally substituted with one to three C₁ to C₃ alkyl groups, a C₁ to C₃ hydroxyalkyl group, and a C₁ to C₆ aminoalkyl group.

76. The composition according to claim 66, wherein Y represents: 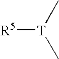
wherein R⁵ represents a polyorganosiloxane chain and T represents a group of formula: 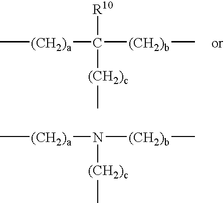
in which a, b and c are, independently, integers ranging from 1 to 10, and R¹⁰ is a hydrogen atom or a group such as those defined for R¹, R², R³ and R⁴, which may be identical or different, represent a group selected from the group consisting of:
linear, branched or cyclic, saturated or unsaturated, C₁ to C₄₀ hydrocarbon-based groups, optionally containing in their chain one or more oxygen, sulphur and/or nitrogen atoms, and possibly being partially or totally substituted with fluorine atoms,
C₆ to C₁₀ aryl groups, optionally substituted with one or more C₁ to C₄ alkyl groups, and
polyorganosiloxane chains possibly containing one or more oxygen, sulphur and/or nitrogen atoms.

77. The composition according to claim 66, wherein R¹, R², R³ and R⁴ represent, independently, a linear or branched C₁ to C₄₀ alkyl group, a polyorganosiloxane chain or a phenyl group optionally substituted with one to three methyl or ethyl groups.

78. The composition according to claim 66, wherein R¹, R², R³ and R⁴ represent, independently, a CH₃, C₂H₅, n-C₃H₇ or isopropyl group,

79. The composition according to claim 61, wherein the structuring polymer comprises at least one moiety of formula: 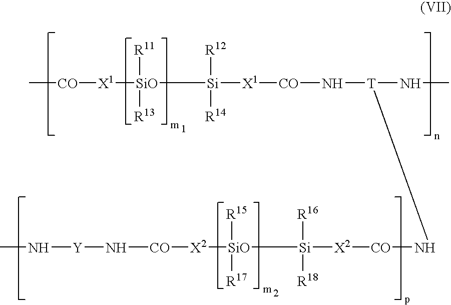
wherein X¹ and X² represent —(CH₂)₁₀—,
n is an integer ranging from 2 to 500,
Y represents —CH₂—, and
T corresponds to one of the following formulae: 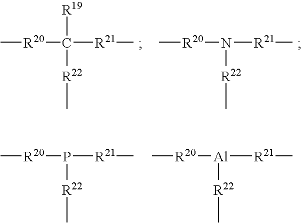
wherein R¹⁹ is a hydrogen atom or a group defined for R¹ to R⁴, and
R²⁰, R²¹ and R²² are, independently, linear or branched alkylene groups,
R¹¹ to R¹⁸ are groups chosen from the same group as R¹ to R⁴,
m₁ and m₂ are numbers in the range from 1 to 1,000, and
p is an integer ranging from 2 to 500.

80. The composition according to claim 79, wherein
p is in the range from 1 to 25,
R¹¹ to R¹⁸ are methyl groups,
T corresponds to one of the following formulae: 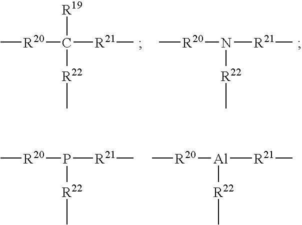
in which R¹⁹ is a hydrogen atom or a group chosen from the groups defined for R¹ to R⁴, and R²⁰, R²¹ and R²² are, independently, linear or branched alkylene groups,
m₁ and m₂ are in the range from 15 to 500,
X¹ and X² represent —(CH₂)₁₀—, and
Y represents —CH₂—.

81. The composition according to claim 80, wherein p is from 1 to 7.

82. The composition according to claim 80, wherein T corresponds to the formula: 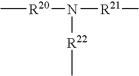
wherein R²⁰, R²¹ and R²² represent —CH₂—CH₂—.

83. The composition according to claim 80, wherein m₁ and m₂ arein the range from 15 to 45.

84. The composition according to claim 66, wherein the polymer comprises at least one moiety corresponding to the following formula: 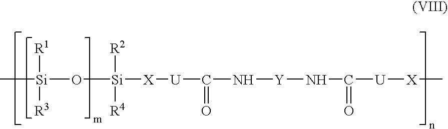
wherein R¹, R², R³, R⁴, X, Y, m and n have the meanings given above for formula (I), and U represents —O— or —NH—, such that: 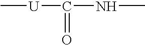
corresponds to a urethane or urea group, or
Y represents:
a C₅ to C₁₂ cycloaliphatic or aromatic group that is optionally substituted with a C₁ to C₁₅ alkyl group or a C₅ to C₁₀ aryl group, or
a linear or branched C₁ to C₄₀ alkylene radical or a C₄ to C₁₂ cycloalkylene radical, or
a polyurethane or polyurea block corresponding to condensing diisocyanate molecules with one or more diol or diamine coupling agents, corresponding to the formula: 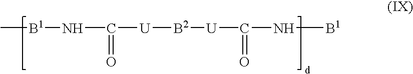
wherein B¹ is a group selected from the group consisting of the groups given above for Y,
U is —O— or —NH—, and
B² is selected from the group consisting of:
linear or branched C₁ to C₄₀ alkylene groups, which can optionally bear an ionizable group such as a carboxylic acid or sulphonic acid group, or a neutralizable or quaternizable tertiary amine group,
C₅ to C₁₂ cycloalkylene groups, optionally bearing alkyl substituents, for example one to three methyl or ethyl groups, or alkylene, for example the diol radical: cyclohexanedimethanol,
phenylene groups that may optionally bear C₁ to C₃ alkyl substituents, and
groups of formula: 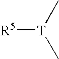
wherein T is a hydrocarbon-based trivalent radical optionally containing one or more hetero atoms selected from the group consisting of oxygen, sulphur and nitrogen; and R⁵ is a polyorganosiloxane chain or a linear or branched C₁ to C₅₀ alkyl chain.

85. The composition according to claim 84, wherein Y represents a C₅ to C₁₂ cycloaliphatic or aromatic group substituted with a radical selected from the group consisting of methylene-4,4-biscyclohexyl radical, a radical of isophorone diisocyanate, a radical of 2,4-tolylenes; a radical of 2,6-tolylenes, 1,5-naphthylene, p-phenylene and 4,4′-biphenylenemethane.

86. The composition according to claim 66, wherein the polymer comprises at least one moiety of formula: 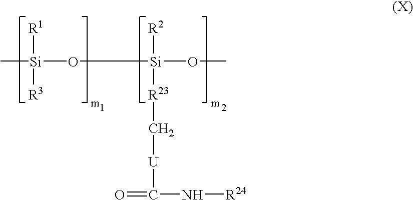
wherein R¹, R², R³, m₁ and m₂ have the meanings given above for formula (I),
U represents O or NH,
R²³ represents a C₁ to C₄₀ alkylene group, optionally comprising one or more O or N hetero atoms, or a phenylene group, and
R²⁴ is a linear, branched or cyclic, saturated or unsaturated C₁ to C₅₀ alkyl group, and a phenyl group optionally substituted with one to three C₁ to C₃ alkyl groups.

87. The composition according to claim 61, wherein the structuring polymer comprises at least one moiety of formula: 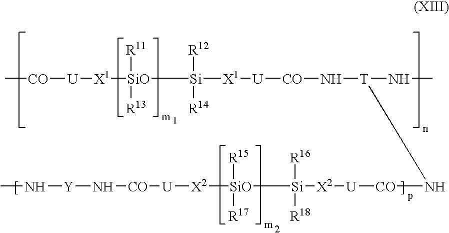
wherein X¹ and X², represent —CH₂)₁₀,
n is an integer from 2 to 500,
Y represents —CH₂—, and
T corresponds to one of the following formulae: 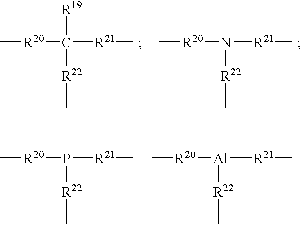
wherein R¹⁹ is a hydrogen atom or a group selected from the group consisting of:
linear, branched or cyclic, saturated or unsaturated, C₁ to C₄₀ hydrocarbon-based groups, optionally containing in their chain one or more oxygen, sulphur and/or nitrogen atoms, and optionally being partially or totally substituted with fluorine atoms,
C₆ to C₁₀ aryl groups, optionally substituted with one or more C₁ to C₄ alkyl groups, and
polyorganosiloxane chains possibly containing one or more oxygen, sulphur and/or nitrogen atoms;
R²⁰, R²¹ and R²² are, independently, linear or branched alkylene groups,
wherein R¹¹ to R¹⁸ are independently selected from the group consisting of:
linear, branched or cyclic, saturated or unsaturated, C₁ to C₄₀ hydrocarbon-based groups, optionally containing in their chain one or more oxygen, sulphur and/or nitrogen atoms, and optionally being partially or totally substituted with fluorine atoms,
C₆ to C₁₀ aryl groups, optionally substituted with one or more C₁ to C₄ alkyl groups, and
polyorganosiloxane chains possibly containing one or more oxygen, sulphur and/or nitrogen atoms;
m₁ and m₂ are numbers from 1 to 1,000, and
p is from 2 to 500.

88. The composition according to claim 66, wherein the structuring polymer further comprises a hydrocarbon-based moiety comprising two groups capable of establishing hydrogen interactions, selected from the group consisting of ester, amide, sulphonamide, carbamate, thiocarbamate, urea, thiourea, oxamido, guanamido and biguanidino, and combinations thereof.

89. The composition according to claim 88, wherein the copolymer is a block copolymer or a grafted copolymer.

90. The composition according to claim 61, wherein the polymer is present in an amount from 0.5% to 80% relative to the total weight of the composition.

91. The composition according to claim 90, wherein the polymer is present in an amount from 2% to 60% relative to the total weight of the composition.

92. The composition according to claim 90, wherein the polymer is present in an amount from 5% to 40% relative to the total weight of the composition.

93. The ccomposition according to claim 61, wherein the at least one structuring polymer has a softening point greater than 50° C.

94. The composition according to claim 61, wherein the at least one structuring polymer has a softening point less than 150° C.

95. The composition according to claim 61, wherein the at least one structuring polymer has a softening point ranging from 70° C. to 130° C.

96. The composition according to claim 61, wherein the at least one structuring polymer has a weight-average molecular mass ranging from 500 to 200,000.

97. The composition according to claim 96, wherein the at least one structuring polymer has a weight-average molecular mass ranging 1,000 to 100,000.

98. The composition according to claim 96, wherein the at least one structuring polymer has a weight-average molecular mass ranging from 2,000 to 30,000.

99. The composition according to claim 61, wherein the composition has a hardness ranging from 30 to 300 gf.

100. The composition according claim 61, which has a hardness ranging from 30 to 250 gf.

101. The composition according to claim 100, which has a hardness ranging from 30 to 200 gf.

102. The composition according claim 61, wherein the at least one liquid fatty phase comprises at least one oil that has an affinity with the least one structuring polymer and selected from the group consisting of at least one polar oil and at least one apolar oil.

103. The composition according to claim 102, which comprises at least one one polar oil and which is selected from the group consisting of:
hydrocarbon-based plant oils with a high content of triglycerides comprising fatty acid esters of glycerol in which the fatty acids comprise chains having from 4 to 24 carbon atoms, said chains optionally being chosen from linear and branched, and saturated and unsaturated chains;
synthetic oils or esters of formula R₅COOR₆ in which R₅ is chosen from linear and branched fatty acid residues comprising from 1 to 40 carbon atoms and R₆ is chosen from hydrocarbon-based chain containing form 1 to 40 carbon atoms, with the proviso that R₅+R₆≧10;
synthetic ethers containing from 10 to 40 carbon atoms;
C₈ to C₂₆ fatty alcohols; and
C₈ to C₂₆ fatty acids.

104. The composition according to claim 102, which comprises at least one apolar oil which is at least one of:
volatile and non-volatile, linear and cyclic polydimethylsiloxanes that are liquid at room temperature;
polydimethylsiloxanes comprising alkyl or alkoxy groups which are pendant and/or at the end of the silicone chain, the groups each containing from 2 to 24 carbon atoms;
phenylsilicones, in particular phenyltrimethicone; and
hydrocarbons chosen from linear and branched, volatile and non-volatile hydrocarbons of synthetic and mineral origin.

105. The composition according to claim 61, wherein the at least one liquid fatty phase is present in an amount ranging from 1% to 99% by weight relative to the total weight of the composition.

106. The composition according to claim 61, wherein the at least one liquid fatty phase is present in an amount ranging from 10% to 80% by weight relative to the total weight of the composition.

107. The composition according to claim 61, wherein the liquid fatty phase comprises more than 30% by weight of at least one silicone-based liquid oil.

108. The composition according to claim 107, wherein the liquid fatty phase comprises more than 40% by weight of at least one silicone-based liquid oil.

109. The composition according to claim 107, wherein the liquid fatty phase comprises from 50% to 100% of at least one silicone-based liquid oil.

110. The composition according to claim 61, wherein the at least one liquid fatty phase comprises at least one volatile solvent selected from the group consisting of a hydrocarbon-based solvent, a silicone solvent, and a mixture thereof.

111. The composition according to claim 110, wherein the at least one liquid fatty phase comprises a silicone solvent comprising alkyl or alkoxy groups that are pendant or at the end of a silicone chain.

112. The composition according to claim 61, wherein the at least one gelling agent is polymeric.

113. The composition according to claim 61, wherein the polymeric gelling agent is selected from the group consisting of crosslinked elastomeric polyorganosiloxanes of three-dimensional structure, polyalkylsesquioxanes, hydrosilylation-reticulated resins, and mixtures thereof.

114. The composition according to claim 61, wherein the polymeric gelling agents are MQ silicone resins.

115. The composition according to claim 114, wherein the polymeric gelling agent comprises hydrophilic groups.

116. The composition according to claim 115, wherein the hydrophilic groups are one or more of polyoxyethylene and copoly(oxyethylene/oxypropylene) groups.

117. The composition according to claim 112, wherein the polymeric gelling agent is selected in the group consisting of the agents that gel via physical reticulation, liposoluble polymers having liquid crystal groups, and mixtures thereof.

118. The composition according to claim 117, wherein the polymeric gelling agent is an agent that gels via physical reticulation selected from the group consisting of molecular muddling, hydrogen interactions, sequences incompatibility, dipolar interactions, and mixtures thereof.

119. The composition according to claim 118, wherein the agents gel via molecular muddling and are silicone gums of formula: 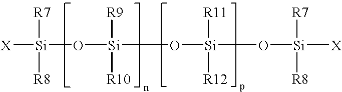
wherein:
R₇, R₈, R₁₁, and R₁₂ are identical or different, and each is an alkyl radical comprising from 1 to 6 carbon atoms,
R₉ and R₁₀ are identical or different, and each is an alkyl radical comprising from 1 to 6 carbon atoms or an aryl radical,
X is an alkyl radical comprising from 1 to 6 carbon atoms, a hydroxyl radical, or a vinyl radical,
n and p are chosen to give the silicone gum a viscosity of greater than 100,000 mPa.s.

120. The composition according to claim 119, wherein n and p are chosen to give the silicone gum a viscosity of greater than 500 000 mPa.s.

121. The composition according to claim 118, wherein the polymeric gelling agent gel via hydrogen interactions and is selected in the group consisting amino silicones polymers having triazinyl groups or pyrimidinyl groups bound to amino groups of amino silicones, non-silicone polyamides which bear ester or triamides functions on an end, polyurethanes, vinyl polymers, (meth)acrylic polymers bearing lateral groups that can create mutual hydrogen interactions, and mixtures thereof.

122. The composition according to claim 118, wherein the polymeric gelling agent gel the liquid fatty phase via sequences incompatibility and is selected from the group consisting of:
di-block copolymers,
tri-block copolymers,
block copolymers comprising a silicone sequence and another sequence that is polyvinyl or poly(meth)acrylic,
grafted copolymers comprising a silicone sequence and another sequence that is polyvinyl or poly(meth)acrylic,
polymers resulting from the polymerization or copolymerization of an ethylenic monomer, comprising one or more ethylenic bonds,
polymers resulting from the polymerization or copolymerization of an ethylenic monomer.

123. The composition according to claim 61, wherein said at least one gelling agent is in mineral form.

124. The composition according to claim 123, wherein said at least one gelling agent is fumed silica.

125. The composition according to claim 61, wherein said at least one gelling agent is present in an amount ranging from 0.05% to 35% by weight relative to the total weight of the composition.

126. The composition according to claim 125, wherein said at least one gelling agent is present in an amount ranging from 0.5% to 20% by weight relative to the total weight of the composition.

127. The composition according to claim 125, wherein said at least one gelling agent is present in an amount ranging from 1% to 10% by weight relative to the total weight of the composition.

128. The composition according to claim 61, further comprising at least one additive selected from the group consisting of antioxidants, essential oils, preserving agents, fragrances, fillers, waxes, neutralizing agents, gums, liposoluble polymers that are dispersible in the medium, cosmetic active agents, dermatological active agents, dispersants, and an aqueous phase containing water that is optionally thickened or gelled with an aqueous-phase thickener or gelling agent and optionally water-miscible compounds.

129. The composition according to claim 61, further comprising at least one coloring agent.

130. The composition according to claim 129, wherein said at least one coloring agent is selected from the group consisting of a lipophilic dye, a hydrophilic dye, a pigment, a nacre, and a mixture thereof.

131. The composition according to claim 129, wherein said at least one coloring agent is present in an amount of from 0.01% to 50% relative to the total weight of the composition.

132. The composition according to claim 131, wherein said at least one coloring agent is present in an amount of from 0.5% to 40% relative to the total weight of the composition.

133. The composition according to claim 131, wherein said at least one coloring agent is present in an amount of from 5% to 30% relative to the total weight of the composition.

134. The composition according to claim 61, which is a solid.

135. The composition according to claim 134, which is a molded stick or poured sticks.

136. The composition according to claim 61, which is in the form of a rigid gel.

137. The composition according to claim 61, which further comprises at least one wax.

138. The composition according to claim 137, wherein said at least one wax is selected from the group consisting of beeswax, carnauba wax, candelilla wax, ouricury wax, Japan wax, cork fibre wax, sugar cane wax, paraffin wax, lignite wax, microcrystalline waxes, lanolin wax, montan wax, ozokerites and hydrogenated oils, polyethylene waxes, waxes obtained by Fischer-Tropsch synthesis, fatty acid esters, glycerides that are solid at 40° C., and silicone waxes, and mixtures thereof.

139. The composition according to claim 61, which is in the form of an anhydrous stick.

140. The composition according to claim 61, which is a mascara, an eyeliner, a foundation, a lipstick, a blusher, a make-up-removing product, a make-up product for the body, an eyeshadow, a face powder, a concealer product, a shampoo, a conditioner, an antisun product or a care product for the lips, skin, or hair.

141. The composition according to claim 61, which is a lipstick.

142. The composition according to claim 61, which is a foundation.

143. The composition according to claim 61, which is anhydrous.

144. A method for care, make-up or treatment of keratin materials comprising applying to the keratin materials the composition according to claim 157.

145. A method for care, make-up or treatment of keratinous fibres, lips, or skin comprising applying to the keratinous fibres, lips, or skin the composition according to claim 61.

146. A method for hardening a make-up composition containing at least one liquid fatty phase comprising
structuring at least one oil with at least one structuring polymer consisting of a polymer with a weight-average molecular mass ranging from 500 to 500,000, containing at least one moiety comprising:
at least one polyorganosiloxane group, consisting of from 1 to 1 000 organosiloxane units in the chain of the moiety or in the form of a graft, and
at least two groups capable of establishing hydrogen interactions, chosen from ester, amide, sulphonamide, carbamate, thiocarbamate, urea, thiourea, oxamido, guanamido and biguanidino groups, and combinations thereof, on condition that at least one of the groups is other than an ester group,
the polymer being solid at 25° C. and soluble in said oil at a temperature of from 25 to 250° C., and
introducing at least one gelling agent for the liquid fatty phase in an amount sufficient to harden the make-up composition.
